(19) United States
(12) Patent Application Publication
(10) Pub. No.: US 2024/0408029 A1
(43) Pub. Date: Dec. 12, 2024

(54) Loading of Extracellular Signaling Molecules Into Lipid-Bound Vesicles for Therapeutic Applications

(71) Applicant: Carnegie Mellon University, Pittsburgh, PA (US)

(72) Inventors: Phil G. Campbell, Cranberry Township, PA (US); Lee E. Weiss, Pittsburgh, PA (US); Saigopalakrishna Saileelaprasad Yerneni, Pittsburgh, PA (US)

(21) Appl. No.: 18/698,912

(22) PCT Filed: Oct. 6, 2022
(86) PCT No.: PCT/IB2022/059569

§ 371 Date: Apr. 5, 2024

Related U.S. Application Data
(60) Provisional application No. 63/252,829, filed on Oct. 6, 2021.

(51) Int. Cl. A61K 9/50; A61K 33/42; A61K 38/18; A61K 38/20; A61K 38/30; A61P 19/00

(52) U.S. Cl. CPC A61K 9/5068; A61K 33/42; A61K 38/1841; A61K 38/1858; A61K 38/1866; A61K 38/1875; A61K 38/20; A61K 38/30; A61P 19/00

(57) ABSTRACT

Provided herein are engineered lipid-bound vesicles for cytosolic delivery of an exogenous secreted, cell surface receptor-binding signaling molecule. Also provided herein are methods of making and using the engineered lipid-bound vesicles, and devices comprising the lipid-bound vesicles.

### CROSS REFERENCE TO RELATED APPLICATIONS

This application is the United States national phase of International Patent Application No. PCT/IB2022/059569 filed Oct. 6, 2022, which claims priority to U.S. Provisional Patent Application No. 63/252,829 filed Oct. 6, 2021, the disclosures of which are incorporated herein by reference in their entireties.

### STATEMENT REGARDING FEDERAL FUNDING

This invention was made with U.S. Government support under AR072954 awarded by the National Institutes of Health (NIH). The U.S. Government has certain rights in this invention.

The Sequence Listing associated with this application is filed in electronic format via EFS-Web and is hereby incorporated by reference into the specification in its entirety. The name of the xml file containing the Sequence Listing is 2205948.xml. The size of the text file is 16,809 bytes, and the xml file was created on Oct. 5, 2022.

Extracellular vesicles (EVs), ranging in size from nanometers (<50 nm) to microns (˜5 μm), are secreted by essentially all cells in the body. EVs are a constituent within the cell microenvironment, occurring both in extracellular body fluids as soluble ‘liquid-phase’ EVs and immobilized to extracellular matrices as ‘solid-phase’ EVs. EVs play a significant role in intercellular communication throughout life, both in health and disease, by acting as delivery vehicles between cells, transporting intraluminal and surface cargo, including DNA, RNA, proteins, lipids, proteoglycans, metabolites, and organelles between cells. Because EVs evolved to deliver cargo, they have gained increasing attention in recent years as vehicles for delivering either native and/or engineered cargo components for therapeutic-specific applications. Encapsulation of both endogenous and exogenous therapeutic agents in the EV lumen protects them from inactivation within the extracellular environment via enzymatic degradation.

Within the complex cargo composition of EVs, numerous growth factors (GFs) have been identified as native EV cargo constituents. Bone morphogenetic proteins (BMPs), transforming growth factor beta (TGFβ), and vascular endothelial growth factors have been identified as constituents in solid-phase EVs immobilized in the extracellular matrix (ECM), while BMP2/4, TGFβ, Wnt proteins, hepatocyte growth factor, and fibroblast growth factor have been found in biological fluids as liquid-phase EV constituents. Although such reports have established that GFs are associated with EVs, the use of EVs as GF delivery vehicles remains relatively unexplored. Naturally occurring EV surface-presented BMP2 can directly interact with their corresponding receptors on the recipient cell membrane and presumably signal intercellularly following endosomal trafficking, similar to EV surface bound TGFβ.

In one example, the clinical application of BMP2 remains challenging, mainly because large pharmacological doses of BMP2 are required due to the poor binding of BMP2 to the collagen type I sponge, which results in burst release and short residence times in vivo. This results in undesirable off-target side-effects, especially when BMP2 is used off-label. As such, improved EV compositions are needed to more safely and effectively deliver BMP2, and other extracellular signaling molecules.

### SUMMARY

A drug-delivery composition is provided comprising an engineered lipid-bound vesicle comprising: a lipid bilayer envelope defining a lumen; and a secreted, cell surface receptor-binding signaling molecule, e.g. comprising a protein or peptide that acts by binding a cell-surface receptor of a target cell, wherein at least 25%, 30%, 40%, 50%, 60%, 75%, 80%, 90%, 91%, 92%, 93%, 94%, 95%, 96%, 97%, 98%, or 99% of the secreted, cell surface receptor-binding signaling molecule is contained within the lumen of the lipid-bound vesicle (that is, not surface bound on the lipid-bound vesicle). A device comprising a substrate and the composition coated on at least a portion of a surface of the substrate and/or incorporated into the substrate also is provided. A method of repairing or producing bone in a patient is provided, comprising administering to the patient at a location of a bone injury or deficit the engineered lipid-bound vesicle in an amount effective to repair of produce bone in the patient, wherein the secreted, cell surface receptor-binding signaling molecule is osteogenic, and optionally osteoinductive (e.g., BMP2 and/or BMP7). A method of inducing a response to an extracellular signaling molecule in a cell is provided, comprising contacting the cell with the engineered lipid-bound vesicle in an amount effective to induce the response in the cell.

A method of preparing an engineered extracellular vesicle loaded with an isolated secreted, cell surface receptor-binding signaling molecule is provided, comprising: sonicating a mixture of an isolated secreted, cell surface receptor-binding signaling molecule with isolated extracellular vesicles, to increase permeability of the extracellular vesicles to the secreted, cell surface receptor-binding signaling molecule, thereby loading the isolated secreted, cell surface receptor-binding signaling molecule into the lumen of the isolated extracellular vesicles, wherein the isolated extracellular vesicles or the loaded extracellular vesicles are stripped of surface-bound secreted, cell surface receptor-binding signaling molecules, for example by acid washing, wherein at least 25%, 30%, 40%, 50%, 60%, 75%, 80%, 90%, 91%, 92%, 93%, 94%, 95%, 96%, 97%, 98%, or 99% of the secreted, cell surface receptor-binding signaling molecule is contained within the lumen of the loaded extracellular vesicle.

The following numbered clauses describe various aspects and embodiments of the present invention.

Clause 1. A drug-delivery composition comprising an engineered lipid-bound vesicle comprising: a lipid bilayer envelope defining a lumen; and a secreted, cell surface receptor-binding signaling molecule, e.g. comprising a protein or peptide that acts by binding a cell-surface receptor of a target cell, wherein at least 25%, 30%, 40%, 50%, 60%, 75%, 80%, 90%, 91%, 92%, 93%, 94%, 95%, 96%, 97%, 98%, or 99% of the secreted, cell surface receptor-binding signaling molecule is contained within the lumen of the lipid-bound vesicle (that is, not surface bound on the lipid-bound vesicle).

Clause 2. The composition of clause 1, wherein the lipid bilayer envelope is an isolated extracellular vesicle (EV) and the secreted, cell surface receptor-binding signaling molecule is exogenous to the EV.

Clause 3. The composition of clause 2, wherein the extracellular vesicle is an isolated exosome, nanovesicle, matrix vesicle, microparticle, or microvesicle obtained from a cell or tissue.

Clause 4. The composition of clause 2 or 3, wherein the extracellular vesicle is obtained from a macrophage or a macrophage cell line.

Clause 5. The composition of any one of clauses 1-4, wherein the secreted, cell surface receptor-binding signaling molecule is a member of the TGF-β superfamily.

Clause 6. The composition of any one of clauses 1-4, wherein the secreted, cell surface receptor-binding signaling molecule is osteogenic, and optionally osteoinductive (e.g., BMP2 and/or BMP7).

Clause 7. The composition of clause 6, wherein the secreted, cell surface receptor-binding signaling molecule is one or more member of a TGF-β family, wherein the bone morphogenic protein is optionally chosen from one of BMP1, BMP2, BMP2A, BMP3, BMP3B, BMP4, BMP5, BMP6, BMP7, BMP8A, BMP8B, BMP9, BMP10, BMP-15, and BMP heterodimers.

Clause 8. The composition of clause 6, wherein the secreted, cell surface receptor-binding signaling molecule is BMP2.

Clause 9. The composition of any one of clauses 1-8, wherein at least 99% of the secreted, cell surface receptor-binding signaling molecule is contained within the lumen of the lipid-bound vesicle.

Clause 10. The composition of any one of clauses 1-9, wherein the lumen of the lipid-bound vesicle comprises at least 1 pg, at least 10 pg, at least 100 pg, at least 500 pg, at least 1 ng, at least 2 ng, at least 3 ng, at least 4 ng, at least 5 ng, at least 6 ng, at least 7 ng, at least 8 ng, at least 9 ng, or at least 10 ng, of the secreted, cell surface receptor-binding signaling molecule per microgram (μg) of total protein of the lipid-bound vesicle.

Clause 11. The composition of any one of clauses 1-10, wherein the lipid-bound vesicle is stripped, for example by washing with an acid or acid buffer (e.g., pH<5 pH) solution, of surface-bound secreted, cell surface receptor-binding signaling molecule.

Clause 12. The composition of any one of clauses 1-11, wherein the lipid-bound vesicle comprises at least two different secreted, cell surface receptor-binding signaling molecules.

Clause 13. The composition of any one of clauses 1-12, comprising a mixture of two different lipid-bond vesicles, each comprising a different secreted, cell surface receptor-binding signaling molecules or different amounts of the secreted, cell surface receptor-binding signaling molecules.

Clause 14. The composition of any one of clauses 1-13, contained within a printer cartridge or reservoir (such as a printer cartridge for an inkjet printer or a 3D printer) or reservoir.

Clause 15. A method of preparing an engineered extracellular vesicle loaded with an isolated secreted, cell surface receptor-binding signaling molecule, comprising: sonicating a mixture of an isolated secreted, cell surface receptor-binding signaling molecule with isolated extracellular vesicles, to increase permeability of the extracellular vesicles to the secreted, cell surface receptor-binding signaling molecule, thereby loading the isolated secreted, cell surface receptor-binding signaling molecule into the lumen of the isolated extracellular vesicles, wherein the isolated extracellular vesicles or the loaded extracellular vesicles are stripped of surface-bound secreted, cell surface receptor-binding signaling molecules, for example by acid washing, wherein at least 25%, 30%, 40%, 50%, 60%, 75%, 80%, 90%, 91%, 92%, 93%, 94%, 95%, 96%, 97%, 98%, or 99% of the secreted, cell surface receptor-binding signaling molecule is contained within the lumen of the loaded extracellular vesicle.

Clause 16. The method of clause 15, comprising: washing surface proteins from isolated extracellular vesicles using an acidic wash solution; separating the isolated extracellular vesicles from the acid wash solution comprising surface proteins eluted from the isolated extracellular vesicles; mixing the acid-eluted extracellular vesicles with the isolated secreted, cell surface receptor-binding signaling molecule; and sonicating the mixture of the isolated secreted, cell surface receptor-binding signaling molecule with the isolated extracellular vesicles obtained from an organism, to increase permeability of the extracellular vesicles to the secreted, cell surface receptor-binding signaling molecule, thereby loading the secreted, cell surface receptor-binding signaling molecules into the lumens of the extracellular vesicles.

Clause 17. The method of clause 15, comprising: mixing the acid-eluted extracellular vesicles with the isolated secreted, cell surface receptor-binding signaling molecule; sonicating the mixture of the isolated secreted, cell surface receptor-binding signaling molecule with the isolated extracellular vesicles obtained from an organism, to increase permeability of the extracellular vesicles to the secreted, cell surface receptor-binding signaling molecule, thereby loading the secreted, cell surface receptor-binding signaling molecules into the lumens of the extracellular vesicles; stripping (washing) surface proteins from the loaded extracellular vesicles using an acidic stripping solution; and separating the loaded extracellular vesicles from the acid wash solution comprising surface proteins eluted from the isolated extracellular vesicles.

Clause 18. The method of any one of clauses 15-17, wherein the extracellular vesicle is an isolated exosome, nanovesicle, matrix vesicle, microparticle, or microvesicle obtained from a cell or tissue.

Clause 19. The method of any one of clauses 15-18, wherein the extracellular vesicle is obtained from a macrophage or a macrophage cell line.

Clause 20. The method of any one of clauses 15-19, wherein the secreted, cell surface receptor-binding signaling molecule is a member of the TGF-β superfamily.

Clause 21. The method of any one of clauses 15-17, wherein the secreted, cell surface receptor-binding signaling molecule is osteogenic, and optionally osteoinductive (e.g., BMP2 and/or BMP7).

Clause 22. The method of clause 21, wherein the secreted, cell surface receptor-binding signaling molecule is one or more of a bone morphogenic protein, TGF-β1, a stromal cell-derived factor (e.g. SDF-1), an insulin-like growth factor (e.g., IGF-2 or IGF-2) or PDGF, wherein the bone morphogenic protein is optionally chosen from one BMP1, BMP2, BMP2A, BMP3, BMP3B, BMP4, BMP5, BMP6, BMP7, BMP8A, BMP8B, BMP9, BMP10, BMP-15, and BMP heterodimers.

Clause 23. The method of clause 21, wherein the secreted, cell surface receptor-binding signaling molecule is BMP2.

Clause 24. The method of any one of clauses 15-23, wherein at least 99% of the secreted, cell surface receptor-binding signaling molecule is contained within the lumen of the loaded extracellular vesicle after stripping.

Clause 25. The method of any one of clauses 15-24, wherein the lumen of the loaded extracellular vesicle comprises at least 500 pg, at least 1 ng, at least 2 ng, at least 3 ng, at least 4 ng, at least 5 ng, at least 6 ng, at least 7 ng, at least 8 ng, at least 9 ng, or at least 10 ng, of the secreted, cell surface receptor-binding signaling molecule per microgram (μg) of total protein of the extracellular vesicle.

Clause 26. The method of any one of clauses 15-25, comprising loading at least two different secreted, cell surface receptor-binding signaling molecules into the isolated extracellular vesicles.

Clause 27. The method of any one of clauses 15-26, further comprising depositing the loaded, stripped extracellular vesicles in a printer cartridge or printer reservoir (such as a printer cartridge or a printer reservoir for use in an inkjet printer or other droplet-based printer, a 3D printer, or an aerosol jet printer).

Clause 28. A device comprising a substrate and the composition of any one of clauses 1-13 coated on at least a portion of a surface of the substrate and/or incorporated into the substrate.

Clause 29. The device of clause 28, wherein the substrate comprises an inorganic material.

Clause 30. The device of clause 29, wherein the inorganic material comprises hydroxyapatite, and the extracellular signaling molecule promotes bone formation.

Clause 31. The device of any one of clauses 28-30, wherein the secreted, cell surface receptor-binding signaling molecule is a TGF-β superfamily ligand (such as a bone morphogenetic proteins or a transforming growth factor beta) or a stromal cell-derived factor (e.g., SDF-1α or SDF-1β), an insulin-like growth factor (e.g., IGF-1 or IGF-2) or a vascular endothelial growth factor (e.g., VEGF-A or VEGF-B) or platelet-derived growth factor (e.g., PDGF-A or PDGF-B) or Interleukin family of cytokines (e.g., IL-1 or IL-2), wherein the bone morphogenetic protein is optionally chosen from one of BMP1, BMP2, BMP2A, BMP3, BMP3B, BMP4, BMP5, BMP6, BMP7, BMP8A, BMP8B, BMP9, BMP10, BMP-15, and BMP heterodimers.

Clause 32. A method of repairing or producing bone in a patient, comprising administering to the patient at a location of a bone injury or deficit an engineered lipid-bound vesicle as described in any one of clauses 1-13 in an amount effective to repair of produce bone in the patient, wherein the secreted, cell surface receptor-binding signaling molecule is osteogenic, and optionally osteoinductive (e.g., BMP2 and/or BMP7).

Clause 33. The method of clause 32, wherein the secreted, cell surface receptor-binding signaling molecule comprises a bone morphogenetic protein (BMP), or TGF-βs, or SDFs, or IGFs, or VEGFs, or PDGFs, or ILs, wherein the BMP is optionally chosen from one of BMP1, BMP2, BMP2A, BMP3, BMP3B, BMP4, BMP5, BMP6, BMP7, BMP8A, BMP8B, BMP9, BMP10, BMP-15, and BMP heterodimers.

Clause 34. The method of clause 32, wherein the secreted, cell surface receptor-binding signaling molecule comprises BMP2 and surface-bound BMP2 is stripped from the engineered EV.

Clause 35. A method of inducing a response to an extracellular signaling molecule in a cell, comprising contacting the cell with the engineered lipid-bound vesicle of any one of clauses 1-13 in an amount effective to induce the response in the cell.

Clause 36. The method of clause 35, wherein the lipid bilayer envelope is an isolated extracellular vesicle (EV) and the secreted, cell surface receptor-binding signaling molecule is exogenous to the EV.

Clause 37. The method of clause 36, wherein the extracellular vesicle is an isolated exosome, nanovesicle, matrix vesicle, microparticle, or microvesicle obtained from a cell or tissue.

Clause 38. The method of clause 36 or 37, wherein the extracellular vesicle is obtained from a macrophage or a macrophage cell line.

Clause 39. The method of any one of clauses 35-38, wherein the secreted, cell surface receptor-binding signaling molecule is a member of the TGF-β superfamily.

Clause 40. The method of any one of clauses 35-39, wherein the secreted, cell surface receptor-binding signaling molecule is osteogenic (BMPs etc.) or osteoinductive (e.g., VEGFs, PDGFs, SDFs, IGFs, ILs etc.).

Clause 41. The method of clause 40, wherein the secreted, cell surface receptor-binding signaling molecule is one or more of a TGF-β family, wherein the bone morphogenic protein is optionally chosen from one of BMP1, BMP2, BMP2A, BMP3, BMP3B, BMP4, BMP5, BMP6, BMP7, BMP8A, BMP8B, BMP9, BMP10, BMP-15, or BMP heterodimers.

Clause 42. The method of clause 40, wherein the secreted, cell surface receptor-binding signaling molecule is BMP2.

Clause 43. The method of any one of clauses 35-42, wherein at least 99% of the secreted, cell surface receptor-binding signaling molecule is contained within the lumen of the lipid-bound vesicle.

Clause 44. The method of any one of clauses 35-43, wherein the lumen of the lipid-bound vesicle comprises at least 1 pg, at least 10 pg, at least 100 pg, at least 500 pg, at least 1 ng, at least 2 ng, at least 3 ng, at least 4 ng, at least 5 ng, at least 6 ng, at least 7 ng, at least 8 ng, at least 9 ng, or at least 10 ng, of the secreted, cell surface receptor-binding signaling molecule per microgram (μg) of total protein of the lipid-bound vesicle.

Clause 45. The method of any one of clauses 35-44, wherein the lipid-bound vesicle is stripped, for example by washing with an acid or acid buffer (e.g., pH<5 pH) solution, of surface-bound secreted, cell surface receptor-binding signaling molecule.

Clause 46. The method of any one of clauses 35-45, wherein the lipid-bound vesicle comprises at least two different secreted, cell surface receptor-binding signaling molecules.

Clause 47. The method of any one of clauses 35-46, comprising a mixture of two different lipid-bond vesicles, each comprising a different secreted, cell surface receptor-binding signaling molecules or different amounts of the secreted, cell surface receptor-binding signaling molecules.

### BRIEF DESCRIPTION OF THE DRAWINGS

FIGS. 1A-1C. EV characterization and loading. (A) ¹²⁵I-BMP2 loading efficiency in EVs using sonication and electroporation techniques. Shown is a representative experiment for 3 independent biological experiments with error bars indicating SEM for three replicates. ***=p≤0.001; ns=no significant difference; AW=acid washed. (B) Nanoparticle analysis of EVs pre- and post-loading using tunable resistive pulse sensing. Sonicated EVs refers to EVs subjected to sonication in the absence of BMP2, eBMP2-EVs refers to EVs sonicated in the presence of BMP2 and native EVs refer to non-sonicated EVs. (C) Representative transmission electron microscopy images of EVs showing vesicular morphology (bars=100 nm). (D) Western blotting characterization of native EVs and eBMP2-EVs. Images show immunoblots for presence of BMP2 and exosome markers CD9, CD63 and TSG101. (E) Retention of ¹²⁵I-BMP2 in EVs in simulated body conditions. Plot indicates the composite of 5 combined experiments. (F) Protection of ¹²⁵I-BMP2 encapsulated in EVs from trypsin and triton X-100. Bars indicate mean±SEM (5 independent experiments), ***=ρ≤0.001.

FIGS. 2A and 2B. In vitro assessment of cellular uptake and signaling by liquid-phase eBMP2-EVs. (A) Flow cytometric analysis of cellular uptake of BMP2, EVs and eBMP2-EVs by MC3T3 cells at 4 hr with and without acid washing (AW). BMP2 was labeled with Alexa Fluor 488 (AF; green) and EVs were labeled with PKH26 (red). (B) Cellular uptake of ¹²⁵I-eBMP2-EVs by C2C12 cells. (C) Representative confocal images showing cellular uptake of fluorescently labeled eBMP2-EVs by C2C12 and MC3T3 cells after 6 hr. Nucleus (blue), EV (red), BMP2 (green) and F-actin (indigo).

FIG. 3. In vitro assessment of liquid-phase eBMP2-EVs bioactivity (A) Bright-field images of ALP assay (C2C12 cells) and mineralization assay (MC3T3 cells). Shown is a representative experiment of 3 independent experiments (B) Dosage curve of eBMP2-EV's bioactivity in ALP assay and mineralization assay. Data represents the mean and SEM for 3 independent experiments; ***=ρ≤0.001; ns=no significant difference vs the no treatment control group. (C) Quantification of ALP staining and alizarin red staining for indicated treatments. Data represents the mean and SEM for 3 independent experiments; ***=ρ≤0.001; ns=no significant difference vs the noggin control group.

FIG. 4. Recycling of BMP2 as nBMP2-EVs from C2C12 cells. (A) Temporal release of internalized ¹²⁵I-BMP2 at 37° C. (B) Bulk of ¹²⁵I-BMP2 release occurs by 30 min, 37° C. (C) Assessment of recycled ¹²⁵I-BMP2 after elution over 2B Sepharose SEC. Fractions 4-6 represented ¹²⁵I-nBMP2-EV while fractions 9-11 represented “free” ¹²⁵I-BMP2. (D) Retention of ¹²⁵I-BMP2 on the EV surface under simulated in vitro body conditions. ¹²⁵I-nBMP2-EVs were incubated at 37° C. and at indicated timepoints samples were eluted over 2B Sepharose SEC to determined ¹²⁵I-BMP2 remaining bound to EV fraction. Cell surface and released distribution of recycled ¹²⁵I-BMP2-EVs. Middle panel-Recycling of ¹²⁵I-BMP2. (E) Immunoprecipitation of recycled ¹²⁵I-BMP2-EV. Aliquots (˜7000 cpm) of recycled ¹²⁵I-BMP2-EV or ¹²⁵I-BMP2 control were immunoprecipitated with either 25 μg of anti-CD81 or with IgG. (F) Cell association of ¹²⁵I-nBMP2-EV after 1 hr, 37° C. (G) Osteogenic activity of recycled BMP2. Unlabeled BMP2 was incubated with C2C12 cells and the 30 min recycled media was collected, pooled, concentrated and eluted over 2B Sepharose SEC. The protein concentration for EV and BMP2 fractions was determined and aliquots of each were assessed for alkaline phosphatase activity compared to eBMP2-EV and control BMP2. Shown is a representative experiment with bars representing mean±SEM for 3 or 6 replicates.

FIGS. 5A-5C. In vitro comparison of liquid-phase BMP2 and eBMP2-EV signaling. (FIG. 5A) qPCR mRNA expression profiles for Dlx3, Runx2, Alpl, and Osx in C2C12 and MC3T3 cells subjected to indicated treatments for 72 hr. Shown is a representative experiment for 2-3 independent biological experiments with error bars indicating SEM for 3 biological replicates. (*=ρ≤0.05, **=ρ≤0.01,***=ρ≤0.001 between compared groups, #=ρ≤0.01 vs BMP2 group) (FIG. 5B) C2C12 and MC3T3 were subjected to indicated treatments for 48 hr and western blot analysis for phospho-SMAD1/5, RUNX2 and β-actin protein expression was evaluated. (FIG. 5C) Relative quantitation of western blot image (FIG. 5B) band intensities relative to β-actin. NPI: Normalized pixel intensity, treatments (a-g) corresponds to respective treatments in (FIG. 5C). Bars indicate mean±SEM (3 independent experiments), *=ρ≤0.05 vs BMP2 group.

FIGS. 6A and 6B. In vitro assessment of liquid-phase eBMP2-EV signaling kinetics. (A) qPCR mRNA expression profiles for Msx2, RunX2, Dlx3, Dlx5, Alpl, and Osx in C2C12 and MC3T3 cells treated with eBMP2-EVs for indicated time-points. (B-C) C2C12 and MC3T3 cells were treated with BMP2 or eBMP2-EVs for indicated time points and levels of total and phosphorylated ERK/p-ERK, p38/p-p38 and β-actin were determined using western blot. Graphs next to the western images represent quantitated band intensities relative to β-actin. Bars indicate mean±SEM (3 independent experiments), NPI: Normalized pixel intensity.

FIGS. 7A and 7B. In vitro and In vivo assessment of solid-phase eBMP2-EVs microenvironments. (A) Bioprinted patterns of Alexa Fluor 488-labeled BMP2 (green) loaded in PKH26-labeled EVs (red). (B) ALP staining of C2C12s post 72 hr seeding on bioprinted patterns with indicated OPs. (C) Quantification of ALP staining of bright-field images shown in FIG. 7B. Shown is a representative experiment for 5 independent biological experiments with error bars indicating SEM. (D) Release kinetics of ¹²¹I-BMP2-EVs from ADM in simulated body fluid for three weeks. (E) Representative μCT 3D reconstructions of mouse leg scans containing either native EV or eBMP2-EVs bioprinted implants. Arrow points to HO. (F) Representative histological images showing H&E and Masson's trichrome staining of native EVs and eBMP2-EVs bioprinted implants (*indicates bone tissue).

FIG. 8. Schematic showing proposed mechanism for BMP2 signaling, cell trafficking, and recycling of BMP2, BMP2 receptors and HSPG, including the formation of nBMP2-EVs. And, the differential signaling via eBMP2-EVs and its role in BMP2 signaling bypassing both cell surface BMP2 receptors and noggin. Numbers within blue circles reflect the order of trafficking events for BMP2 signaling, beginning with receptor binding (1) through to recycling (5). nBMP2-EV is subject to signaling via the BMP2 pathway. Numbers within orange circles reflect the order of trafficking events for eBMP2-EV signaling, beginning with receptor binding (1) through to recycling (6). Arrows provide additional directional cues. Blue arrows indicate trafficking pathway, black arrows indicate signaling pathway and red arrows indicate delivery to lysosome degradation pathway.

FIG. 9. Table contrasting eBMP2-EVs to nBMP2-EVs.

### DETAILED DESCRIPTION

The use of numerical values in the various ranges specified in this application, unless expressly indicated otherwise, are stated as approximations as though the minimum and maximum values within the stated ranges are both preceded by the word “about.” In this manner, slight variations above and below the stated ranges can be used to achieve substantially the same results as values within the ranges. Also, unless indicated otherwise, the disclosure of these ranges is intended as a continuous range including every value between the minimum and maximum values.

As used herein, the terms “comprising,” “comprise” or “comprised,” and variations thereof, are meant to be open ended. The terms “a” and “an” are intended to refer to one or more.

Provided herein is a method for loading a secreted, cell surface receptor-binding signaling molecule, such as a growth factor, directly into lumen of a lipid-bound vesicle, e.g., an extracellular vesicle (EVs), which can enable a manner of therapeutic delivery that minimizes extracellular proteolysis, inhibition of the influence of inhibitory proteins, and bypasses surface receptors. The method permits the molecules to bypass their cell surface receptors to signal target cells, which is in direct contrast to established signaling dogma, such as growth factor signaling dogma. In some embodiments, lipid-bound vesicles, such as isolated EVs can be loaded with secreted, cell surface receptor-binding signaling molecules via sonication. In some embodiments, once internalized into EVs, secreted, cell surface receptor-binding signaling molecules can be entrapped by, surrounded by, and protected by the EV plasma membrane. The lipid-bound vesicle may comprise a growth factor, such as bone morphogenetic protein 2 (BMP2) or bone morphogenetic protein 7 (BMP7). Also provided herein is a composition comprising the lipid-bound vesicle.

The methods and compositions may be applicable to any secreted, cell surface receptor-binding signaling molecule, such as a hormone, cytokine, or growth factor, across a broad range of tissue engineering and regenerative medicine therapeutic applications. In an example, BMP7 can be loaded into the lipid-bound vesicle and may be used to treat a wound, that is in wound healing to promote scar-less healing and immune-acceptance during organ transplantation, as well as in an anti-cancer therapy.

As used herein, “treatment” or “treating” refers to administration to a patient by any suitable dosage regimen, procedure and/or administration route of a composition, device or structure as described herein with the object of achieving a desirable clinical/medical end-point, including healing a wound, correcting a defect, ameliorating cancer, etc.

As used herein, the term “patient” or “subject” refers to members of the animal kingdom including but not limited to human beings and “mammal” refers to all mammals, including, but not limited to human beings.

Described herein are engineered lipid-bound vesicles comprising: a lipid bilayer envelope defining a lumen; and a secreted, cell surface receptor-binding signaling molecule, e.g. a protein or peptide that acts by binding a cell-surface receptor of a target cell, contained predominantly within the lumen of the lipid bilayer envelope, such as at least 25%, 30%, 40%, 50%, 60%, 75%, 80%, 90%, 91%, 92%, 93%, 94%, 95%, 96%, 97%, 98%, or 99% of the secreted, cell surface receptor-binding signaling molecule is contained within the lumen of the lipid-bound vesicle (that is, not surface bound on the lipid-bound vesicle). Where the lipid-bound vesicle is a naturally-occurring lipid-bound vesicle, such as an EV isolated or otherwise prepared from a cell or tissue, the secreted, cell surface receptor-binding signaling molecule is exogenous, meaning that it is added to the lumen of the vesicle, where the secreted, cell surface receptor-binding signaling molecule is either different from naturally-occurring secreted, cell surface receptor-binding signaling molecule present in the EV, or the secreted, cell surface receptor-binding signaling molecule is present in the EV but additional quantities of the secreted, cell surface receptor-binding signaling molecule are loaded into the lumen of the EV. By “secreted”, it is not implied that the as-loaded signaling molecule was manufactured by secretion, but that the signaling molecule is normally secreted in a patient. As such, the molecule, such as BMP2, may be manufactured by any useful synthetic method, including, for example and without limitation, by solid-phase peptide synthesis or recombinant methods. The lipid-bound vesicle may be isolated EVs prepared from a eukaryotic cell (e.g. an isolated cell, a cell line, a tissue, or an organ), such as a mammalian or human cell, cell line, tissue, or organ, such as a monocyte cell or cell line, for example and without limitation a macrophage cell or cell line. Typical lipid-bound vesicles prepared from EVs comprise protein content in addition to the extracellular signaling molecule, such as surface receptors and ligands. The lipid-bound vesicle may be stripped of surface-bound extracellular signaling molecule, e.g., by acid washing, for example at pH 3.0, such that the surface of the lipid-bound vesicle is free of, or substantially free of (e.g. no more than 3%, 2%, 1%, or 0.1% of total extracellular signaling molecule within or on the lipid-bound vesicle) the extracellular signaling molecule. The lipid bound vesicles comprise exogenous extracellular signaling molecule in amounts effective to elicit a desired physiological response, such as specific gene expression patterns, cell growth, cell differentiation, or cell response in cells, such as inducing expression, production, or secretion of a gene product. For example, the lumen of the lipid-bound vesicle comprises at least 1 pg, at least 10 pg, at least 100 pg, at least 500 pg, at least 1 ng, at least 2 ng, at least 3 ng, at least 4 ng, at least 5 ng, at least 6 ng, at least 7 ng, at least 8 ng, at least 9 ng, or at least 10 ng, of the secreted, cell surface receptor-binding signaling molecule per microgram (μg) of total protein of the lipid-bound vesicle.

In another aspect a method of making an engineered lipid-bound vesicle is described, in which EVs are isolated from a eukaryotic cell (e.g., an isolated cell, a cell line, a tissue, or an organ), such as a mammalian or human cell or cell line, such as a mammalian or human monocyte cell or cell line, for example and without limitation a mammalian or human macrophage cell or cell line. Next, exogenous extracellular signaling molecule is loaded into the lumen of the EVs, for example, by sonication. Prior to or after loading the exogenous extracellular signaling molecule into the lumen of the EVs, surface-bound factors, such as surface-bound extracellular signaling molecules may be stripped from the surface of the EVs, such that no extracellular signaling molecules, or substantially no extracellular signaling molecules (e.g., no more than 3%, 2%, 1%, or 0.1% of total extracellular signaling molecule within or on the lipid-bound vesicle) remain surface-bound to the EVs. For example, after loading the exogenous extracellular signaling molecule into the lumen of the EVs, surface-bound factors, such as surface-bound extracellular signaling molecules are stripped from the surface of the EVs, such that no extracellular signaling molecules, or substantially no extracellular signaling molecules (e.g., no more than 3%, 2%, 1%, or 0.1% of total extracellular signaling molecule within or on the lipid-bound vesicle) remain surface-bound to the EVs. Surface-bound factors are readily stripped from the surface of the EVs by acid washing, which typically involves bringing the solution in which the EVs are contained to an acidic pH, e.g. of from 2.0 to 4.0, and for example pH=3, and separating by any physical means the stripped factors from the EV, such as by size exclusion, or pelleting the EVs, removing the supernatant or fraction containing the stripped factors, and suspending the stripped EVs in a suitable solution, such as water, PBS, normal saline, etc. Exemplary acid pH solutions include, without limitation: HCl, pH 2.0-4.0; HCl-glycine, pH 3.0; acetic acid 2.0-4.0; and acidic conditions with and without 150 mM NaCl. Solutions used for stripping may be referred to herein as “stripping solution” or “stripping buffer” which may be an aqueous solution comprising an acid, an acidic buffer, and/or salts, having a pH of <7, <6, <5, <4, or any useful range therein so long as the solution is able to remove surface-bound factors from the vesicles without damaging vesicle function for the intended use. Other methods of stripping surface-bound factors include: removal via proteolytic digestion (without limitation: trypsin; protease K) or charge interaction/exchange (for example and without limitation: heparin; NaCl). The engineered lipid-bound vesicle may be deposited on or into a substrate by any useful means. In one example, the engineered lipid-bound vesicles are inkjet-printed, or otherwise drop-wise printed, on a surface of a substrate. The engineered vesicles may be co-deposited with a substrate material and thereby integrated into the substrate. The engineered vesicles may be adsorbed onto or absorbed into a porous or open cell or porous substrate or non-woven material. The engineered vesicles may be deposited or stored in a printer cartridge or a printer reservoir, for example and without limitation, for use in an inkjet printer or other droplet-based printer, a 3D printer, or an aerosol jet printer.

Also provided herein are devices comprising a substrate and the engineered vesicles as described herein on or in the substrate, for example on a surface of the substrate or within the substrate. The substrate may be any substrate as described herein, such as a cell growth scaffold, and may comprise a polymer, ceramic, or other biocompatible material as described herein, such as a bioerodable polymer, a non-bioerodable polymer, and/or a calcium phosphate material, such as hydroxyapatite. In one example, the substrate is a 3D-printed bioerodable polymer configured to repair a bone or tissue injury.

Provided herein is a method of repairing or producing bone in a patient, comprising administering to the patient at a location of a bone injury or deficit, e.g., due to a congenital defect, an engineered lipid-bound vesicle, such as an engineered EV loaded with exogenous BMP2 and/or BMP7, in which surface-bound growth factors, including any surface-bound BMP2 and/or BMP7 are stripped from the engineered EV. The engineered EVs may be delivered as part of a device comprising a substrate, such as a cell-growth scaffold comprising the engineered EVs associated therewith, e.g., adsorbed onto, absorbed into, and/or deposited on or in the substrate. For example, the substrate may be printed to a 3D shape configured to repair a defect or injury to a bone, and the engineered EVs may be printed onto and/or into the substrate to promote bone growth. As above, the substrate may be polymeric, ceramic, e.g., comprising a bioerodable polymer and/or a calcium phosphate material such as hydroxyapatite. The device may be placed, screwed, or glued in place, for example bone cement, e.g., polymethyl methacrylate adhesive, may be used to anchor the device at the site of a bone repair.

Provided herein is a method of delivering an extracellular signaling molecule, such as a growth factor to a cell, comprising contacting the cell with an engineered lipid-bound vesicle as described herein. The engineered EVs may be delivered as part of a device comprising a substrate, such as a cell-growth scaffold comprising the engineered EVs associated therewith, e.g. adsorbed onto, absorbed into, and/or deposited on or in the substrate. For example, the substrate may be printed to a 3D shape configured to repair a defect or injury to a tissue, and the engineered EVs may be printed onto and/or into the substrate to promote tissue growth. As above, the substrate may be polymeric, ceramic, e.g., comprising a bioerodable polymer and/or a calcium phosphate material such as hydroxyapatite. The device may be placed, sutured, screwed, or glued in place, for example using a fibrin glue, an acrylic adhesive, or bone cement, e.g., polymethyl methacrylate adhesive, may be used to anchor the device at the site of a bone repair.

In addition to the cell-derived or tissue-derived EV's, engineered lipid-bound vesicles may be prepared synthetically (see, e.g., Li, Y J., Wu, J Y., Liu, J. et al. Artificial exosomes for translational nanomedicine. J Nanobiotechnol 19. 242 (2021) as an example of synthetic lipid-bound vesicle or EVs. Such synthetic lipid-bound vesicles may be loaded with a secreted, cell surface receptor-binding signaling molecule as described herein, e.g., by sonication, and any surface-bound secreted, cell surface receptor-binding signaling molecules on the lipid-bound vesicle may be stripped from the synthetic lipid-bound vesicle as described herein.

As used herein, the term “polymer composition” is a composition comprising one or more polymers. As a class, “polymers” includes, without limitation, homopolymers, heteropolymers, copolymers, block polymers, block co-polymers and can be both natural and synthetic. Homopolymers contain one type of building block, or monomer, whereas copolymers contain more than one type of monomer.

A polymer “comprises” or is “derived from” a stated monomer if that monomer is incorporated into the polymer. Thus, the incorporated monomer that the polymer comprises is not the same as the monomer prior to incorporation into the polymer, in that at the very least, during incorporation of the monomer, certain groups, e.g., terminal groups, that are modified during polymerization are changed, removed, and/or relocated, and certain bonds may be added, removed, and/or modified. An incorporated monomer is referred to as a “residue” of that monomer. A polymer is said to comprise a specific type of linkage if that linkage is present in the polymer. Unless otherwise specified, molecular weight for polymer compositions refers to weight average molecular weight (MW). A “moiety” is a portion of a molecule, compound or composition, and includes a residue or group of residues within a larger polymer.

A bioerodible polymer is a polymer that degrades in vivo over a time period, which can be tailored to erode over a time period ranging from days to months, and up to two years, for example a polymeric structure, when placed in vivo, will fully degrade within a time period of up to two years. By “bioerodible,” it is meant that a polymer, once implanted and placed in contact with bodily fluids and/or tissues, will degrade either partially or completely through chemical, biochemical and/or enzymatic processes. Non-limiting examples of such chemical reactions include acid/base reactions, hydrolysis reactions, and enzymatic cleavage. In certain non-limiting embodiments, the biodegradable polymers may comprise homopolymers, copolymers, and/or polymeric blends comprising, without limitation, one or more of the following monomers: glycolide, lactide, caprolactone, dioxanone, and trimethylene carbonate. In other non-limiting embodiments, the polymer(s) comprise labile chemical moieties, non-limiting examples of which include esters, anhydrides, or polyanhydrides, which can be useful in, for example and without limitation, controlling the degradation rate of the structure, such as a cell-growth scaffold or particles and/or the release rate of therapeutic agents, such as the modified lipid-bound vesicles, e.g., EVs, from the scaffold or particles.

By “biocompatible,” it is meant that a polymer composition and its normal degradation in vivo products are cytocompatible and are substantially non-toxic and non-carcinogenic in a patient within useful, practical and/or acceptable tolerances. By “cytocompatible,” it is meant that the polymer can sustain a population of cells and/or the polymer composition, device, and degradation products thereof are not cytotoxic and/or carcinogenic within useful, practical and/or acceptable tolerances. For example, the polymer when placed in a human cell culture does not adversely affect the viability, growth, adhesion, and number of cells. In one non-limiting embodiment, the compositions and/or devices are “biocompatible” to the extent they are, for example acceptable for use in a human or veterinary patient according to applicable regulatory standards in a given jurisdiction. In another example the biocompatible polymer, when implanted in a patient, does not cause a substantial adverse reaction or substantial harm to cells and tissues in the body, for instance, the polymer composition or device does not cause unacceptable inflammation, necrosis, or an infection resulting in harm to tissues from the implanted structure.

Non-limiting examples of a bioreodible polymer useful for substrates, e.g., tissue or cell growth scaffolds, described herein, include: a polyester, a polyester-containing copolymer, a polyanhydride, a polyanhydride-containing copolymer, a polyorthoester, and a polyorthoester-containing copolymer. In one aspect, the polyester or polyester-containing copolymer is a poly(lactic-co-glycolic) acid (PLGA) copolymer. In another embodiment, the bioerodible polymer is selected from the group consisting of poly(lactic acid) (PLA); poly(trimethylene carbonate) (PTMC); poly(caprolactone) (PCL); poly(glycolic acid) (PGA); poly(glycolide-co-trimethylenecarbonate) (PGTMC); poly(L-lactide-co-glycolide) (PLGA); polyethylene-glycol (PEG-) containing block copolymers; and polyphosphazenes. Additional bioerodible, biocompatible polymers include: a poly(ester urethane) urea (PEUU); poly(ether ester urethane)urea (PEEUU); poly(ester carbonate)urethane urea (PECUU); poly(carbonate)urethane urea (PCUU); a polyurethane; a polyester; a polymer comprising monomers derived from alpha-hydroxy acids such as: polylactide, poly(lactide-co-glycolide), poly(L-lactide-co-caprolactone), polyglycolic acid, poly(dl-lactide-co-glycolide), and/or poly(l-lactide-co-dl-lactide); a polymer comprising monomers derived from esters including polyhydroxybutyrate, polyhydroxyvalerate, polydioxanone, and/or polyglactin; a polymer comprising monomers derived from lactones including polycaprolactone; or a polymer comprising monomers derived from carbonates including polycarbonate, polyglyconate, poly(glycolide-co-trimethylene carbonate), or poly(glycolide-co-trimethylene carbonate-co-dioxanone).

Non-bioerodable polymers either do not erode substantially in vivo or erode over a time period of greater than two years. Compositions such as, for example and without limitation, PTFE, poly(ethylene-co-vinyl acetate), poly(n-butylmethacrylate), poly(styrene-b-isobutylene-b-styrene) and polyethylene terephthalate are considered to be non-erodable polymers. Other suitable non-erodable polymer compositions are broadly known in the art, for example in stent coating and transdermal reservoir technologies. The growth structures described herein may comprise a non-erodible polymer composition.

Natural polymers, such as natural polypeptides, polysaccharides, or glycoproteins may be used in the formation of a substrate, including, for example, collagen (e.g., collagen sponge) or a more complex extracellular matrix (ECM) material, such as decellularized ECM. Polysaccharides, such as alginate, cellulose, or other biocompatible polysaccharides may be included in the substrate.

A “substrate” in the context of the present disclosure is a two- or three-dimensional structure having a surface, onto which the engineered lipid-bound vesicles described herein may be deposited, absorbed onto, adsorbed into, or otherwise associated with the structure. The surface may be on the structure, or within the structure, for example on an outward-facing portion of the structure, on or within channels or pores within the structure, or internal to a dissolvable or bioerodable structure, to be exposed on dissolution or bioerosion/biodegradation of the substrate over time. A substrate may comprise, for example and without limitation, a polymer, such as a bioerodable polymer, a non-bioerodable polymer, a mixture of bioerodable and non-bioerodable polymers, or a ceramic. The substrate may be a polymeric cell-growth scaffold. The substrate may comprise a calcium phosphate, such as hydroxyapatite. The substrate may be a collagen matrix or sponge. The substrate may be a non-woven material, for example, prepared by electrodeposition, or a molded structure. The substrate may be formed by 3D printing, and may recapitulate a natural structure of a patient, for example based on imaging data, e.g., x-ray or MRI data, obtained from a patient. The substrate may be a cell culture vessel, such as a dish, flask, suspended particles, or a bioreactor of any size.

Secreted, cell surface receptor-binding signaling molecules are soluble molecules found in multicellular organisms, such as mammals and humans, that secreted by cells and travel extracellularly to bind receptors on the surface of a target cell to act on that target cell, and can include, for example and without limitation growth factors, cytokines, and hormones. Extracellular signaling molecules may be peptides or small-molecule compounds. They include autocrine, paracrine, juxtacrine and endocrine signals. By their nature they exclude signaling across gap junctions (e.g., intracellular mediators) and juxtacrine signaling where the ligands are both membrane-associated and will present on the surface of the engineered EVs described herein. They also exclude signaling molecules, such as steroids, that pass through the cell membrane to bind internal receptors in cells. Secreted, cell surface receptor-binding signaling molecules can stimulate cell growth, differentiation, survival, inflammation, and/or tissue repair.

The signal transduction of secreted extracellular signaling molecules typically is initiated by binding to their receptors on the surface of target cells. The information conveyed by a growth factor to a particular subpopulation of cells depending on the type of receptors, number of target cell, and the intracellular signal transduction subsequent to factor binding. External factors such as the binding ability of an extracellular signaling molecule to extracellular matrices (ECM), ECM degradation, and concentration of the extracellular signaling molecule may have an effect on the ultimate response of a target cell to a specific extracellular signaling molecule. Presented herein is a novel “stealth” mechanism for activation of cells by extracellular signaling molecules by first delivering those molecules into the cytoplasm of the target cells, bypassing canonical direct receptor-mediated signaling by the extracellular signaling molecules, and preventing inhibitory effects by factors that can sequester/bind to the signaling molecule and prevent its binding to the cell surface receptor, such as noggin for BMP2. The membrane-bound vesicles, e.g. EVs or engineered vesicles, described herein may be preferably stripped of any surface-bound extracellular signaling molecules to completely side-step direct surface receptor-mediated binding and signaling by any extracellular signaling molecules presenting on the surface of the engineered lipid-bound vesicles.

Non-limiting examples of extracellular signaling molecule include growth factors that interact with specific receptors at the cell surface and includes, for example and without limitation: bone morphogenic protein (BMP), epidermal growth factor (EGF), insulin-like growth factor (IGF), growth hormone (somatotropin), stromal cell-derived growth factor (SGF), platelet-derived growth factors (PDGF), and fibroblast growth factors (FGFs). The extracellular signaling molecule may be a member of the transforming growth factor-beta (TGFB1) superfamily of regulatory molecules, including, for example and without limitation, bone morphogenetic proteins (BMPs), growth and differentiation factors (GDFs), activins (ACTs), inhibins (INHs), and glial-derived neurotrophic factors (GDNFs) (See, e.g., Poniatowski ŁA, et al., Transforming growth factor Beta family: insight into the role of growth factors in regulation of fracture healing biology and potential clinical applications. Mediators Inflamm. 2015; 2015:137823; Wrana J L. Signaling by the TGFβ superfamily. Cold Spring Harb Perspect Biol. 2013 Oct. 1; 5(10):a011197; and Hanna A, et al., The Role of the TGF-β Superfamily in Myocardial Infarction. Front Cardiovasc Med. 2019 Sep. 18; 6:140). An exemplary list of extracellular signaling molecules includes: TGF-β1, TGF-β2, BMP2, BMP6, BMP7, BMP9, IGF-1, IGF-2, EGF, PDGF-AA, PDGF-BB, PDGF-AB, FGF1, FGF2 through FGF23, hepatocyte growth factor (HGF), interleukins, vascular endothelial growth factor (VEGF), Wnts, neurotophins, Ephrins, and stromal-derived cell factor (SDF). The previous individually-identified molecules, and classes of molecules, including human versions thereof, such as BMPs or members of the TGF-β superfamily, are broadly-known and described in the literature and in public sequence databases, including, without limitation GenBank and UniProt, and many, if not all, are commercially-available from a variety of sources.

By “osteogenic” in reference to a growth factor or other signaling molecule, it is meant a compound that promotes bone formation in a patient by itself or in combination with another growth factor. As demonstrated herein and elsewhere, BMP2 is osteogenic. Other bone morphogenic proteins are osteogenic. Other growth factors also are osteogenic, e.g., osteoinductive. Non-limiting examples of osteogenic growth factors include: BMP2, BMP3, BMP6, BMP7, BMP8A, BMP8B, TGF-β1, and PDGF, where BMP2 and BMP7 are osteoinductive.

Extracellular vesicles (EVs), ranging in size from nanometers (<50 nm) to microns (˜5 μm), are secreted by essentially all cells in the body. EVs are a constituent within the cell microenvironment, occurring both in extracellular body fluids as soluble ‘liquid-phase’ EVs and immobilized to extracellular matrices as ‘solid-phase’ EVs. EVs play a significant role in intercellular communication throughout life, both in health and disease, by acting as delivery vehicles between cells, transporting intraluminal and surface cargo, including DNA, RNA, proteins, lipids, proteoglycans, metabolites, and organelles between cells. Because EVs evolved to deliver cargo, they have gained increasing attention in recent years as vehicles for delivering either native and/or engineered cargo components for therapeutic-specific applications. Encapsulation of both endogenous and exogenous therapeutic agents in the EV lumen protects them from inactivation within the extracellular environment via enzymatic degradation.

The examples below provide proof of concept of the usefulness of the described engineered lipid-bound vesicles. While proof of concept was demonstrated using BMP2, the described compositions are expected to effectively deliver other secreted, cell surface receptor-binding signaling molecules directly to the cytosol of a target cell, bypassing direct cell surface receptor action, yet eliciting similar physiological effect as if the molecules directly stimulated their cognate cell surface receptor. Preliminary results indicate that IGF-1 can be loaded into EVs and current work includes optimization of loading and delivery of IGF-1.

### Example—Cell Trafficking and Regulation of Osteoblastogenesis by Extracellular Vesicle Associated Bone Morphogenetic Protein 2

Extracellular vesicles (EVs) are characterized by complex cargo composition and carry a wide array of signaling cargo, including growth factors (GFs). Beyond surface-associated GFs, it is unclear if EV intralumenal growth factors are biologically active. Here, bone morphogenetic protein-2 (BMP2), loaded directly into the lumen of EVs designated engineered BMP2-EVs (eBMP2-EVs), was comprehensively characterized including its regulation of osteoblastogenesis. eBMP2-EVs and non-EV “free” BMP2 were observed to similarly regulate osteoblastogenesis. Furthermore, cell trafficking experiments suggest rapid BMP2 recycling and its extracellular release as “free” BMP2 and natural occurring BMP2-EVs (nBMP2-EVs), with both being osteogenic. Interestingly, BMP2 occurs on the EV surface of nBMP2-EVs and is susceptible to proteolysis, inhibition by noggin and complete dissociation from nBMP2-EVs over three days. Whereas, within the eBMP2-EVs, BMP2 is protected from proteolysis, inhibition by noggin and is retained in EV lumen at 100% for the first 24 hours and ˜80% after ten days. Similar to “free” BMP2, bioprinted eBMP2-EV microenvironments induced osteogenesis in vitro and in vivo in spatial registration to the printed patterns. Taken together, BMP2 signaling involves dynamic BMP2 cell trafficking in and out of the cell involving EVs, with distinct differences between these nBMP2-EVs and eBMP2-EVs attributable to the BMP2 cargo location with EVs. Lastly, eBMP2-EVs appear to deliver BMP2 directly into the cytoplasm, initiating BMP2 signaling within the cell, bypassing its cell surface receptors.

In further detail, within the complex cargo composition of EVs, numerous growth factors (GFs) have been identified as native EV cargo constituents. Bone morphogenetic proteins (BMPs), transforming growth factor beta (TGFβ) and vascular endothelial growth factors have been identified as constituents in solid-phase EVs immobilized in the extracellular matrix (ECM), while BMP2/4, TGFβ, Wnt proteins, hepatocyte growth factor, and fibroblast growth factor have been found in biological fluids as liquid-phase EV constituents. Although such reports have established that GFs are associated with EVs, the use of EVs as GF delivery vehicles remains relatively unexplored. In regard to BMP2, Draebing et al. reported that the transport of BMP2/4 by EVs is an essential mechanism for developmental morphogenesis (Draebing, T., et al., Extracellular vesicle-delivered bone morphogenetic proteins: A novel paracrine mechanism during embryonic development. bioRxiv (2018)). Wei et al. demonstrated that EVs secreted by BMP2-treated macrophages exhibited dramatically improved osteogenic bioactivity in vitro, although they did not determine whether BMP2 was an EV cargo constituent (Wei, F., et al. Exosome-integrated titanium oxide nanotubes for targeted bone regeneration. Acta Biomater 86, 480-492 (2019)). Huang et al. genetically modified bone marrow-derived mesenchymal stem cells to overexpress BMP2 and found that although secreted EVs did not contain BMP2, EVs were enriched in multiple osteogenic miRNAs (Huang, C. C., et al. Functionally engineered extracellular vesicles improve bone regeneration. Acta Biomater 109, 182-194 (2020)). Others have engineered EVs to deliver BMP2 plasmid DNA. Naturally-occurring EV surface-presented BMP2 can directly interact with their corresponding receptors on the recipient cell membrane and presumably signaling intercellularly following endosomal trafficking, similar to EV surface bound TGFβ. However, the potential pathway(s) mediating EV intraluminal GF signaling remain unclear. Understanding such signaling pathway(s) could potentially shed light on alternate mechanisms by which EV encapsulated intraluminal GFs control cell fate.

Beyond diffusible soluble EVs, ECM immobilized, solid-phase EVs likely enable persistent tissue-specific microenvironments that spatially localize their cell signaling cargo. Such microenvironments would therefore be relevant to tissue maintenance and normal functioning, similar to how native (non-EV-associated), solid-phase GFs in the ECM are involved in tissue homeostasis. Therefore, recapitulating not only native solid-phase EV microenvironments but also those incorporating exogenous GF-EVs may biomimick localized delivery of GF-based therapeutics. Here, we report an extension of our bioprinting technology to create spatially defined solid-phase EV-based microenvironments (Yerneni, S. S., et al., Bioprinting exosome-like extracellular vesicle microenvironments. Bioprinting, e00041 (2019) and Yerneni, S. S., et al., Rapid On-Demand Extracellular Vesicle Augmentation with Versatile Oligonucleotide Tethers. ACS Nano 13, 10555-10565 (2019)), consisting of engineering EVs with intralumenally loaded exogenous BMP2 (eBMP2-EVs).

Recombinant human BMP2 was selected as a paradigm GF due to its biological and clinical significance. The clinical application of BMP2 remains challenging, mainly because large pharmacological doses of BMP2 are required due to the poor binding of BMP2 to the collagen type I sponge, which results in burst release and short residence times in vivo. This results in undesirable off-target side-effects, especially when BMP2 is used off-label. Potential advantages of utilizing EV delivery vehicles for BMP2 include: (i) the high binding capacity of EVs for ECM constituents, including collagen type 17, and (ii) the ability of EVs to protect intraluminal cargo from extravesicular antagonists/inhibitor/enzyme degradation. Therefore, eBMP2-EVs may act as a BMP2 depot, requiring lower doses that might mitigate off-target effects. Beyond the basic science aspects, this report lays the groundwork toward establishing and validating a methodology to engineer and deliver solid-phase EVs containing intraluminal exogenous GFs. This work expands our previous studies using bioprinted spatially controlled solid-phase non-EV-associated GF microenvironments which include extensive in vitro and in vivo application of bioprinted BMP2 (Cooper, G. M., et al. Inkjet-based biopatterning of bone morphogenetic protein-2 to spatially control calvarial bone formation. Tissue Eng Part A 16, 1749-1759 (2010); Phillippi, J. A., et al. Microenvironments engineered by inkjet bioprinting spatially direct adult stem cells toward muscle- and bone-like subpopulations. Stem Cells 26, 127-134 (2008); Miller, E. D., et al. Inkjet printing of growth factor concentration gradients and combinatorial arrays immobilized on biologically-relevant substrates. Comb Chem High Throughput Screen 12, 604-618 (2009); and Tuzmen, C., et al., Crosstalk between substance P and calcitonin gene-related peptide during heterotopic ossification in murine Achilles tendon. J Orthop Res 36, 1444-1455 (2018)). We originally pioneered this biopatterning technology based on biomimicry to engineer microenvironments with protein and peptide-based signaling molecules, and their response modifiers, to recapitulate aspects of biological spatial patterning of cell functions occurring during morphogenesis and tissue repair/regeneration. This approach has the potential to accelerate the application of EVs delivering GFs as practical therapeutics, particularly those based on localized solid-phase delivery.

Here, both liquid- and solid-phase eBMP2-EVs were studied. Loading efficiency, retention, and protection of exogenously loaded BMP2 in EVs was assessed using radiolabeling-based assays. Radiolabeling, fluorescence, and gene/protein expression assays were performed to validate liquid-phase eBMP2-EV cell trafficking and osteogenic bioactivity in vitro. Moreover, the ability of eBMP2-EVs to protect BMP2 from noggin, a natural BMP2 inhibitor, was also assessed. Cell trafficking experiments using “free” BMP2 (BMP2 not associated with EVs) confirmed that BMP2 incorporation into EV cargo occurs naturally, however, unlike eBMP2-EVs, this naturally cell loaded form of BMP2-EVs (nBMP2-EVs), had the BMP2 unstably localized on the EV surface. Moreover, this EV surface presented BMP2 was inhibited by noggin. Bioprinting was used to create solid-phase eBMP2-EV microenvironments on collagen-coated coverslips for in vitro studies and in collagen-rich acellular dermal matrix (ADM) material for in vivo studies. In vitro, picogram level doses of intraluminal EV BMP2 in eBMP2-EVs induced osteogenic differentiation in registration to bioprinted patterns. In vivo, nanogram level doses induced localized heterotopic ossification (HO) in a mouse muscle pocket model. Taking the in vitro experiments together, the data suggest that BMP2 cell signaling involves cell trafficking whereby cell internalized BMP2 is recycled as EV cargo, but that the nBMP2-EVs exhibits distinct attributes compared to eBMP2-EVs. The data further suggests a novel intracellular signaling mechanism for eBMP2-EVs beyond the established GF signaling paradigm which is initiated by a GF binding to its cell surface receptor.

### Materials and Methods

EV nomenclature: Over the past 50 years investigators have referred to extracellular vesicles using dozens of different classification criteria for naming different types of vesicles released from prokaryotic and eukaryotic cells, such as matrix vesicles, exosomes, nanovesicles, microvesicles and microparticles, just to name a few. However, since the myriad of identified types were based on different criteria and outdated isolation and detection techniques, the single term ‘EV’ was introduced by International Society on Extracellular Vesicles (ISEV) to avoid confusion and to improve the exchange of information between investigators and societies.

Cell culture: Mouse J774A.1 cells (ATTC® TIB-67™, Manassas) were grown and maintained in Roswell Park Memorial Institute medium (RPMI, Gibco, Gaithersburg, MD) supplemented with 10% heat-inactivated fetal bovine serum (HI-FBS; Invitrogen, Carlsbad, CA) and 1% penicillin-streptomycin (PS; Invitrogen, Carlsbad, CA). Mouse C2C12 cells (ATCC® CRL-1772™, Manassas, VA) were grown in Dulbecco's Modified Eagle's Media (DMEM; Invitrogen, Carlsbad, CA) containing 10% HI-FBS and 1% penicillin-streptomycin. MC3T3-E1 subclone 4 cells (ATCC® CRL-2593™, Manassas, VA) were grown in ascorbic acid-free α-minimum essential media (αMEM, Gibco, Gaithersburg, MD) media supplemented with 10% HI-FBS and 1% PS. In all cell culture experiments, EV-depleted FBS obtained by centrifugation at 100,000×g for 2 hr was utilized. Conditioned media was collected every 72 hr and stored at −80° C. if not used immediately for EV isolation.

Isolation of EVs: EVs from conditioned media were isolated by size exclusion chromatography (SEC). Briefly, conditioned media was centrifuged at 2,000×g for 10 min at 4° C. and then at 10,000×g for 30 min at 4° C. Supernatant was passed through a 0.22 μm-pore Millipore filter and EVs isolated by mini-SEC using 1.5 cm×12 cm mini-columns (Bio-Rad, Hercules, CA, USA; Econo-Pac columns) packed with 10 ml of Sepharose 2B (Sigma-Aldrich, St. Louis, MO, USA) equilibrated with phosphate-buffered saline (PBS). Supernatant (1.0 ml) was loaded onto the column and five 1 ml fractions corresponding to the void volume peak were collected by running PBS over the column. Fraction 4 was used for subsequent experiments. Isolated EVs were either used immediately (within 24 hr) for subsequent experiments or stored at −80° C. for long term storage.

Characterization of EVs: EVs were characterized by quantifying the protein content, transmission electron microscopy (TEM), tunable resistive pulse sensing (TRPS) and western blotting (see section on immunoblotting) for EV (exosome) surface markers as described previously and according to the current MISEV2018 guidelines (Thery, C., et al. Minimal information for studies of extracellular vesicles 2018 (MISEV2018): a position statement of the International Society for Extracellular Vesicles and update of the MISEV2014 guidelines. J Extracell Vesicles 7, 1535750 (2018)). EV concentrations are reported in μg/ml EV protein. Detailed experimental procedures are included in the supplemental information.

Iodination of BMP2: Recombinant human BMP2 (Medtronic, Inc., Minneapolis, MN) was iodinated via the chloramine T method (Miller, E. D., et al. Inkjet printing of growth factor concentration gradients and combinatorial arrays immobilized on biologically-relevant substrates. Comb Chem High Throughput Screen 12, 604-618 (2009)) as adapted from the original iodination protocol established for TGFβ (Frolik, C. A., et al., Characterization of a membrane receptor for transforming growth factor-beta in normal rat kidney fibroblasts. J Biol Chem 259, 10995-11000 (1984)). BMP2 (10 pg) was reacted with 500 μCi ¹²⁵I-Na at 25° C. with stepwise addition of 3 aliquots of dilute chloramine T solution (100 μg/ml). The resulting ¹²⁵I-BMP2 was >97% trichloroacetic acid perceptible with minimal protein aggregate formation. Specific activity of ¹²⁵I-BMP2 was from 55-80 μCi/μg.

Loading of BMP2 into EVs: Two different physical loading methods, electroporation and sonication, were investigated for loading BMP2 into EVs. A mixture of 10 μg of EVs and 1 pg BMP2 were used for these experiments. For electroporation, the mixture was electroporated (Bio-Rad Laboratories, Hercules, CA) at 1 kV for 5 ms, and then incubated at 37° C. for 1 hr to allow membrane recovery. For sonication, the mixture was sonicated (Tekmar sonic disruptor) on ice using a 0.25″ tip at 20% amplitude, 6 cycles of 30 s on/off for 3 min with a 2 min cooling period between each cycle. The unloaded BMP2 was removed using a 100,000 kDa MWCO membrane filter (Vivaspin® columns, Sartorius AG, Göttingen, Germany). EV surface-bound BMP2 was removed by pH 3.0 acid-incubation followed by separation of EVs from BMP2 using mini-SEC. Loading of BMP2 in EVs was confirmed by immunoblotting and ¹²⁵I-BMP2.

Retention of BMP2 in EVs: ¹²⁵I-BMP2 was loaded into EVs using the procedures described above. Approximately 50 μl of ¹²⁵I-BMP2 corresponding to ˜1×10⁷ cpm was mixed with 1 μg of unlabeled BMP2. Immediate post-loading and purification, aliquots of ¹²⁵I-BMP2/BMP2/EVs containing ˜100,000 cpm ¹²⁵I-BMP2 were aliquoted into 12×75 mm polypropylene tubes containing simulated body fluid (SBF; composition: 10% FBS, 0.02% sodium azide, 25 mM HEPES in DMEM) to a total 1 ml volume. Tubes were incubated at 37° C. and samples were eluted using Sepharose 2B SEC with PBS at 23° C. at different time points, with a primary focus on early time points but going out to 14 days.

Radioactivity associated with EV and protein fractions were determined. The percent ¹²⁵I-BMP2 retained in EVs was expressed as the percentage of radioactivity associated with the EV peak over the combined EV and protein peaks. The zero timepoint was determined immediately post purification and data was normalized to percent ¹²⁵I-BMP2 retained in EVs. To confirm intralumenal localization of ¹²⁵I-BMP2 in EVs, ¹²⁵I-eBMP2-EVs were treated with 1 mM trypsin, for a minimum of 30 min, 37° C., then eluted over 2B Sepharose. The resulting radioactivity associated with the EV fraction was compared to the EV fraction of a non-protease control group. The proteolytic sensitivity of “free” ¹²⁵I-BMP2 was confirmed by treating ¹²⁵I-BMP2 with trypsin under similar conditions, then conducting TCA precipitation to access degradation. ¹²⁵I-eBMP2-EVs were also treated with 0.1% Triton-X100 for a minimum of 15 min, 25 C, followed by SEC to confirm detergent-based disruption of ¹²⁵I-eBMP2-EVs.

Radiolabeled-based cell binding and cell trafficking experiments: Confluent cultures of C2C12 cells in 6-well plates were rinsed twice with PBS and incubated in 1 ml cell binding buffer (0.1 M HEPES 0.12 M NaCl, 5 mM KCl, 1.2 mM MgSO₄, 8 mM glucose, 1% BSA, pH 7.4 under 37° C. in CO₂ cell culture conditions for 1 hr. Buffer was aspirated and fresh binding buffer was added along with ˜100,000 cpm of ¹²⁵I-eBMP2-EVs. At indicated time points, representative plates were placed on ice, buffer was aspirated and cells were rinsed three times with ice-cold PBS. 1 ml ice-cold acid wash buffer (50 mM glycine HCl, 01 M NaCl, pH 3) was added to each well and cells were incubated on ice for 10 min. Acid buffer was collected into 12×75 mm polypropylene tubes for radioactive determination. This represented the portion of ¹²⁵I-eBMP2-EVs bound to the cell surface. NaOH (1 M) was added at 1 ml per well and cells incubated with agitation for 30 min at RT. Solubilized cells were then transferred to 12×75 mm polypropylene tubes for radioactive determination. This represented the portion of ¹²⁵I-eBMP2-EVs internalized within cells. Data was presented as total cell associated, cell surface and cell internalized radioactivity.

¹²⁵I-BMP2 or ¹²⁵I-eBMP2-EVs were associated with cells as described above, excepting that once the acid rinse step was collected, cells containing internalized ¹²⁵I-BMP2 or ¹²⁵I-eBMP2-EVs were transitioned from ice to 37° C. incubation with fresh cell binding buffer. After 30 min at 37° C., the cells were again placed on ice and the buffer was collected for counting, which represents the recycled ¹²⁵I-BMP2, ¹²⁵I-nBMP2-EVs or ¹²⁵I-eBMP2-EVs that is released from the cell intracellular compartment into the buffer. The retained cell layer was incubated with ice-cold acid rinse buffer for 10 min, 4° C. The acid wash was collected for counting, which represents the recycled ¹²⁵I-BMP2, ¹²⁵I-nBMP2-EVs or ¹²⁵I-eBMP2-EVs bound to the cell surface. This by convention is used to also represent the recycled BMP2 “receptors.” The remaining intracellular radioactivity was determined using 1 N NaOH. Recycling data was presented as either percent of internalized ¹²⁵I-BMP2, ¹²⁵I-nBMP2-EVs or ¹²⁵I-eBMP2-EVs recycled, and the percent of cell surface or released recycled radioactivity. Trichloroacetic acid (TCA) precipitation was performed on recycled buffer to assess degradation of cell trafficked ¹²⁵I-BMP2. Specifically regarding ¹²⁵I-BMP2, in some experiments the released recycled radioactivity was pooled after initial counting, concentrated using a 3 kDa spin filter, the passed over SEC to separate the EV and protein fractions. The radioactivity associated with the EV fraction was expressed as recycled ¹²⁵I-nBMP2-EV. Aliquots of recycled ¹²⁵I-nBMP2-EVs were accessed for their ability to bind and be internalized in C2C12 cells. Aliquots were also immunoprecipitated against anti-CD81 to confirm the association of radioactivity with EVs. Additionally, unlabeled 100 ng BMP2 was incubated with C2C12 cells and 30 min recycled nBMP2-EV and BMP2 protein fraction was derived. The osteogenic ability of recycled BMP2 was assessed using alkaline phosphatase assay.

Osteogenic differentiation assays: Alkaline Phosphatase (ALP) Assay—C2C12 cells were incubated with indicated treatments, washed with PBS to remove culture medium, and fixed for 20 min with 10% neutral buffered formalin (Millipore-Sigma). Alkaline phosphatase activity was detected using a leukocyte alkaline phosphatase assay kit according to the manufacturer's instructions (Millipore-Sigma, St. Louis, MO). Where required, ALP-stained images were converted to CMYK format since this color format is representative of reflected light colors as opposed to emitted light colors (RGB). Since the combination of cyan and magenta form the color blue, these channels were added together and inverted. The average pixel intensity was determined using the image histogram tool in Adobe® Photoshop 7.0 (Adobe® Systems, San Jose, CA).

Mineralization Assay—MC3T3-E1 (subclone 4) cells were seeded in growth media (ascorbic acid-free α-MEM, 10% FBS, 1% PS). After 24 hr post-seeding, differentiating media (alpha-MEM, 10% FBS, 50 μg/ml ascorbic acid, 10 mM β-glycerophosphate, 1% PS) was added with indicated treatments. Media (supplemented with respective treatments) was changed every 72 hr. On day 21, cells were fixed in 10% neutral buffered formalin, washed with distilled water three times, and alizarin red stain (Millipore-Sigma, St. Louis, MO) was added to the wells and incubated for 1 hr at RT. After imaging the cells, quantification of mineralization was performed using an osteogenesis quantitation kit (Millipore-Sigma, St. Louis, MO) according to manufacturer's instructions. Briefly, alizarin red stained cells were treated with 10% acetic acid solution for 30 min with shaking, cells were scraped, centrifuged and the dissolved alizarin stain was quantified by measuring the OD at 405 nm (TECAN plate reader, Mannedorf, Switzerland) using alizarin red reference standards.

Real-time quantitative PCR (qPCR): RNA from MC3T3 and C2C12 cells was isolated using a RNeasy Mini Kit (Qiagen, Hilden, Germany) according to the manufacturer instructions. First-Strand cDNA Synthesis System (Life Technologies, Carlsbad, CA) was used for cDNA synthesis. qPCR was carried out using 2× Maxima SYBR Green/ROX qPCR Master Mix (Thermo Fisher Scientific, Waltham, MA) in Fast 96-Well Reaction Plates (Applied Biosystems, Foster City, CA) using a StepOnePlus (Applied Biosystems, Foster City, CA). Relative mRNA levels were calculated using the ΔΔCt method using 18srRNA for normalization. The qPCR primers are listed in Table 1.

TABLE 1

| Gene | Forward primer (5′→3′) | Reverse primer (5′→3′) |
|---|---|---|
| 18srRNA | GAGCGACCAAAGGAACCATA | CGCTTCCTTACCTGGTTGAT |
| | (SEQ ID NO: 1) | (SEQ ID NO: 2) |
| Runx2 | CCTCTGACTTCTGCCTCTGG | ATGAAATGCTTGGGAACTGC |
| | (SEQ ID NO: 3) | (SEQ ID NO: 4) |
| Ocn | TAGTGAACAGACTCCGGCGCTA | TGTAGGCGGTCTTCAAGCCAT |
| | (SEQ ID NO: 5) | (SEQ ID NO: 6) |
| Bsp | AAGAAGAGGAAGAGGAAGAAAATGA | GCTTCTTCTCCGTTGTCTCC |
| | (SEQ ID NO: 7) | (SEQ ID NO: 8) |
| Osx | AGAGGTTCACTCGCTCTGACGA | TTGCTCAAGTGGTCGCTTCTG |
| | (SEQ ID NO: 9) | (SEQ ID NO: 10) |
| Alp1 | CACGGCCATCCTATATGGTAA | CTGGGCCTGGTAGTTGTT |
| | (SEQ ID NO: 11) | (SEQ ID NO: 12) |
| Dlx3 | GTACCGGGAGCAGCCTTT | CTTCCGGCTCCTCTTTCA |
| | (SEQ ID NO: 13) | (SEQ ID NO: 14) |
| Dlx5 | GCCCCTACCACCAGTACG | TCACCATCCTCACCTCTG |
| | (SEQ ID NO: 15) | (SEQ ID NO: 16) |
| Msx2 | ATACAGGAGCCCGGCAGATA | CGGTTGGTCTTGTGTTTCCT |
| | (SEQ ID NO: 17) | (SEQ ID NO: 18) |

Immunoblotting: Immunoblotting was performed according to previously published protocols (Adamik, J., et al. EZH2 or HDAC1 Inhibition Reverses Multiple Myeloma-Induced Epigenetic Suppression of Osteoblast Differentiation. Mol Cancer Res 15, 405-417 (2017)) using the following antibodies directed towards: rhBMP2 (Abcam, 214821), RUNX2 (CST, D1L7F), p-SMAD1/5-S463/465 (CST, 41D10), TSG101 (Abcam, 125011), CD63 (Abcam, ab216130), CD9 (Abcam, 192726), β-ACTIN (Sigma, 85316), p38 (SC, sc-535), p-p38-Thr180/Tyr182 (CST, 4511), ERK (Santa Cruz, sc-94) and p-ERK1/2-Thr2O2/Tyr2O4 (CST, 4370). Band signals were detected using Amersham ECL Western Blotting Detection Reagent (GE Life Sciences, Marlborough, MA) and analyzed and quantitated using ProteinSimple Imager and AlphaView software (ProteinSimple, San Jose, CA).

In vivo assessment in a mouse muscle pocket model: C57BL/6 male mice (n=5; 22-26 grams) were utilized for evaluation of ADM scaffolds bioprinted with eBMP2-EVs or EVs alone implanted in a murine muscle pocket. eBMP2-EVs or native EVs were printed at a total concentration of 150 ng EVs (containing 5 ng BMP2) per 4.5 mm ADM disc. Post bioprinting, overnight rinsing was performed to wash-off unbound EVs prior to the implantation. Surgeries on each mouse were performed on both hind legs, with one leg serving as a non-printed control. The thigh areas were first shaved to remove hair then treated with antiseptic agent (Povidone iodine 10%, Clinipad Corporation, Guilford, CT) followed by 70% ethyl alcohol. The thigh muscle on the dorsal side (Biceps femoris) was surgically exposed and a pocket was made in that muscle using a pair of forceps. ADM alone or ADM bioprinted with eBMP2-EVs was folded in half, inserted into the pocket and a single suture (Proline® 6-0) was used to close the opening of the pocket. The wound was closed with interrupted sutures. The treatments (eBMP2-EVs or EV alone controls) were randomized in each leg. Following the surgery, animals were given 1 mg/ml pediatric ibuprofen (Rite Aid, Camp Hill, PA) for pain through their drinking water. Locomotion, grooming, and eating habits of the animals were monitored post-surgery. Animals were euthanized at four weeks via CO₂ inhalation. For tissue harvest, the skin was removed from the legs, which were separated from the mouse at the hip sockets and fixed in 10% buffered formalin for 4 days at RT prior to μCT analysis.

Tunable resistive pulse sensing (TRPS): TRPS system by qNano (Izon, Cambridge, MA,) was used to measure the size distribution and concentration of particles in isolated EV fractions as previously described1. 40 μl EV suspension or calibration particles included in the reagent kit (2:1, 114 nm, Izon) were placed in the Nanopore (NP100 #A28126, Izon). All samples were measured at 45.06 mm stretch at 0.64 V and 11 mbar pressure. Particles were detected in short pulses of the current (blockades). The calibration particles were measured directly before and after the experimental sample under identical conditions. The sizes and concentrations of particles were determined using software provided by Izon (version 3.2).

Transmission Electron Microscopy: TEM characterization was performed as previously described1. Briefly, isolated total EVs were fixed with 4% glutaraldehyde (Electron Microscopy Services, Hatfield, PA) for 20 min at RT. A 10 μL droplet of glutaraldehyde-fixed EVs was placed on Formvar-coated 300 mesh copper grid (Electron Microscopy Services, Hatfield, PA). The sample was incubated for 1 min followed by rinsing with distilled water for 1 min to ensure removal of PBS salts. Excess liquid was blotted-off with a Whatman filter. Post rinsing, 50 μl of Uranyl-acetate solution was put on the grid and allowed to remain for 1 min. Excess liquid was removed, and the grids were viewed on a Hitachi H-7100 transmission electron microscope (TEM, Hitachi High Technologies) operating at 100 keV. Digital images were collected using an AMT Advantage 10 CCD Camera System (Advanced Microscopy Techniques) and inspected using NIH ImageJ software.

Flow cytometry tracking: BMP2 was labelled with Alexa Fluor 647 using a Microscale Protein Labeling Kit (Thermo Fisher Scientific, Waltham, MA). PKH26-labeled EVs were loaded with Alexa Fluor 647-labeled BMP2. MC3T3 cells were treated with Alexa Fluor 647-BMP2, PKH26-EVs or eBMP2-EVs for 4 hr and then analyzed for green and red fluorescence, with and without acid rinsing. Flow cytometric analysis was performed on an Accuri C6 flow cytometer (BD Biosciences, San Jose, CA) connected to an Intellicyt HyperCyt autosampler (IntelliCyt Corp., Albuquerque, NM) using green (488 nm) and red (649 nm) channels. Data were analyzed using FlowJo® software (Flowjo LLC, Ashland, Oregon).

Confocal microscopy: PKH26-labeled EVs were loaded with Alexa Fluor 647-labeled BMP2 and incubated with MC3T3 cells for designated time points. To remove plasma membrane-bound EVs, cells were treated with stripping buffer (500 μM NaCl and 0.5% acetic acid in deionized water, pH: 3) for 45 seconds followed by three washes with PBS. Cells were fixed with 3.33% freshly prepared paraformaldehyde (Electron Microscopy Services, Hatfield, PA) for 20 min at room temperature (RT). Excess fixative was quenched by adding an equal volume of 1% (w/w) BSA in PBS for 5 min followed by three washes with PBS. Fixed cells were permeabilized with 0.1% Triton X-100 in PBS for 1 min. To visualize F-actin and nuclei, cells were stained with Alexa Fluor 488-Phallodin (5:200 in PBS; Thermo Fisher Scientific, Waltham, MA) and Hoechst 33342 (1:1000 in PBS; Thermo Fisher Scientific, Waltham, MA), respectively. Imaging was performed using a Carl Zeiss LSM 880 confocal microscope with fixed settings across all of the experimental time points, and the images were analyzed using ZEN Black software (Carl Zeiss Microscopy, Thornwood, NY).

Bioprinting: Bioprinting of eBMP2-EVs was accomplished using our previously established inkjet-based bioprinting system (Yerneni, S. S., et al. Bioprinting exosome-like extracellular vesicle microenvironments. Bioprinting, e00041 (2019)). A piezoelectric drop-on-demand printhead with a diamond-like carbon-coated 60 μm diameter nozzle (MicroFab Technologies, Inc., Plano, TX) was used for these experiments. A dilute EV bioink consisted of 100 μg/ml eBMP2-EVs (0.018 ng BMP2/ng EV) in PBS with 10% glycerol. All the inks were degassed for 10 min prior to printing. The concentration of deposited BMP-EVs at individual locations on printing substrates or scaffolds was modulated using an overprinting strategy whereby the concentration increases with the number of overprints (OPs). To validate the printing process, defined patterns of fluorescently labeled eBMP2-EVs (Alexa Fluor 647-labeled BMP2 and PKH26-labeled EVs) were printed on collagen type I coated coverslips (Neuovitro, Vancouver, WA) as 1.25×1.75 mm patterns arranged in 2×2 dose-modulated arrays of 5, 10, 15, and 20 OPs, with adjacent drop spacings of 80 μm.

For in vitro biological response experiments, collagen type I coated coverslips were printed as described above but with 10, 20, 30, and 40 OPs. After printing, the coverslips were rinsed with PBS overnight at 4° C. before seeding with C2C12 cells. After 3 days under cell culture conditions, coverslips were stained for ALP activity. For the solid-phase in vivo studies, 50 OPs of eBMP2-EVs or sonicated EVs without BMP2 controls were uniformly printed on 200 μm thick, 4.5 mm diameter discs of ADM (DermaMatrix™, MTF Foundation, Edison, NJ), where the bioinks were absorbed into the discs. We have used this thin, collagen-rich material in prior feasibility bioprinting studies using small animal models in order to accommodate implantation into defects.

Micro-computed tomography (pCT) analysis: Bone formation was analyzed using a VivaCT 40 (SCANCO Medical AG, Bassersdorf, Switzerland) μCT system. The femur and tibia, with surrounding soft tissue, were imaged in 70% ethyl alcohol inside a holder provided by the manufacturer and stabilized with parafilm. Imaging was performed using a 30 μm voxel size and the following conditions: 55 kVp/145 μA, FOV/Diameter of 30 mm, and single frame-averaging. 3D reconstruction of the scanned volumes with raw files was done automatically by the system's operating software. The SCANCO μCT 3D morphometry and analysis software, which operates in an open VMS environment, was used for analysis. Region of interests (ROIs) were drawn by tracing the borders of the heterotopic bone formed around the thigh region where the ADM scaffolds were implanted, and a global threshold of 158.4 mg HA/ccm was applied for the heterotypic bone/background segmentation. The thresholding value represented the peak characteristic of bone tissue in the gray value distribution histogram as previously described5. The ROI regions were assessed for bone volume (BV).

Decalcification and histology: After micro-CT analysis, legs were placed in nylon mesh bags (Electron Microscopy Sciences, Hatfield, PA) and subjected to Cal-Rite™ solution (Richard-Allan Scientific™, San Diego, CA) at RT for two weeks with constant agitation. The thigh region consisting of the implant was sectioned and embedded in paraffin blocks for histological analysis. The paraffin embedded specimens were sectioned at a thickness of 5 μm and stained with hematoxylin and eosin and Masson's trichrome stain.

Statistics: Data are presented as the mean±SEM (n is as indicated in figure legends). One-way analysis of variance (ANOVA) was used for data analysis to determine any statistically significant differences between two and multiple groups with Tukey's post-hoc analysis where appropriate using GraphPad Prism (v8.0) software. P≤0.05 was considered significant.

### Results

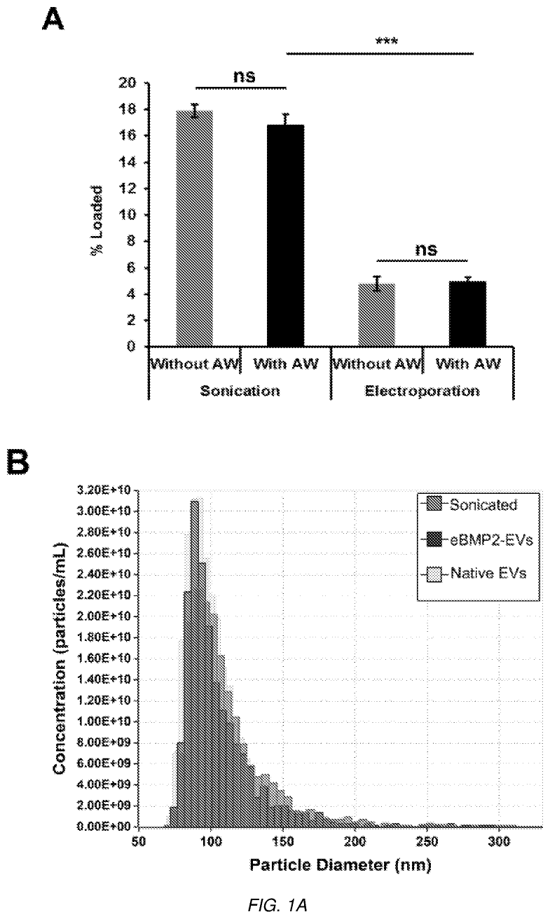

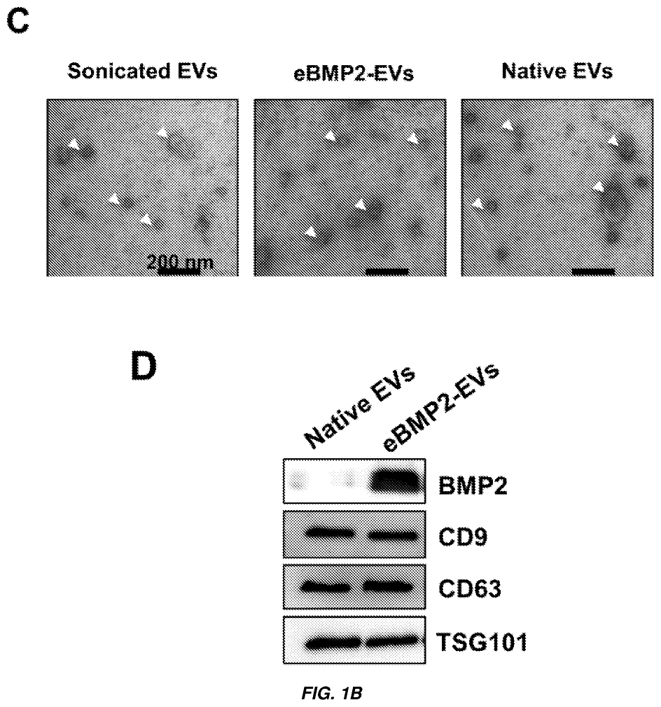

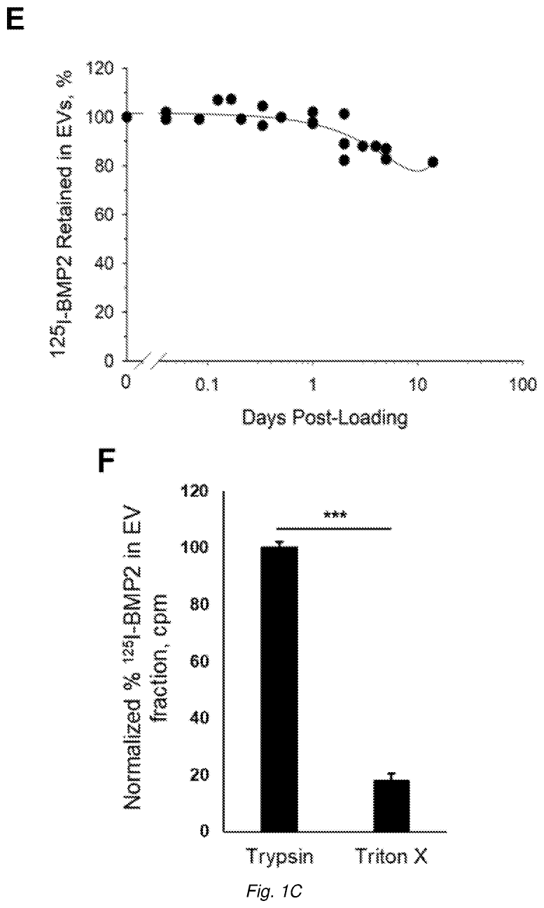

EV isolation, loading and characterization: The murine J774A.1 monocytic cell line, in its inactivated Mo state, served as our source of EVs. The average size of EVs, isolated from J774A.1 cells, was 100 nm and the EV protein concentration was 75-100 μg/ml. BMP2 was loaded into EVs either by electroporation or sonication (FIG. 1A (A)). To evaluate the loading efficiency of BMP2 into EVs, ¹²¹I-BMP2 was utilized. Acid buffer washing post-loading was performed to release any external membrane-bound BMP2, leaving only internalized intraluminal BMP2. BMP2 loading efficiencies were 18±1.9% with sonication and 5±0.7% with electroporation (FIG. 1A (A)). Since sonication resulted in over 3-fold higher loading efficiency, sonication was utilized for BMP2-EV formulation for all subsequent experiments. Tunable resistive pulse sensing (TRPS) analysis revealed that there was no drastic change in EV size distribution profile between pre- and post-sonication (FIG. 1A (B)). Transmission electron microscopy (TEM) analysis demonstrated no difference between pre-sonication native EVs and sonicated EVs with and without BMP2 (FIG. 1B (C)). Exosome markers TSG101, CD63 and CD9 were identified via western blot analysis and were similar between native EVs and eBMP2-EVs (FIG. 1B (D)). Western blot analysis of BMP2 demonstrated the presence of BMP2 only in eBMP2-EVs (FIG. 1B (D)). The retention of BMP2 in eBMP2-EVs was evaluated using ¹²⁵I-BMP2. Essentially 100% of BMP2 was retained within the EV lumen during the first 24 hr of incubation in simulated body fluid (SBF) at 37° C. with a gradual loss thereafter. Approximately 80% of the loaded BMP2 was retained in the EVs after 10 days post loading (FIG. 1C (E)). The loaded eBMP2-EVs were also subjected to protease (trypsin) treatment followed by separation using size exclusion chromatography (SEC) to further confirm that the BMP2 in eBMP2-EVs was intraluminal. We observed complete protection of EV-encapsulated BMP2 from trypsin, whereas >95% of “free” BMP2 was degraded based on TCA precipitation (FIG. 1C (F)). Detergent treatment with Triton-X 100 further confirmed that BMP2 was contained within EVs. These data suggest that our loading protocol results in intraluminal loading of BMP2 in EVs and demonstrate successful non-cell loading of BMP2 within cell secreted EVs, with minimal impact on measured EV properties.

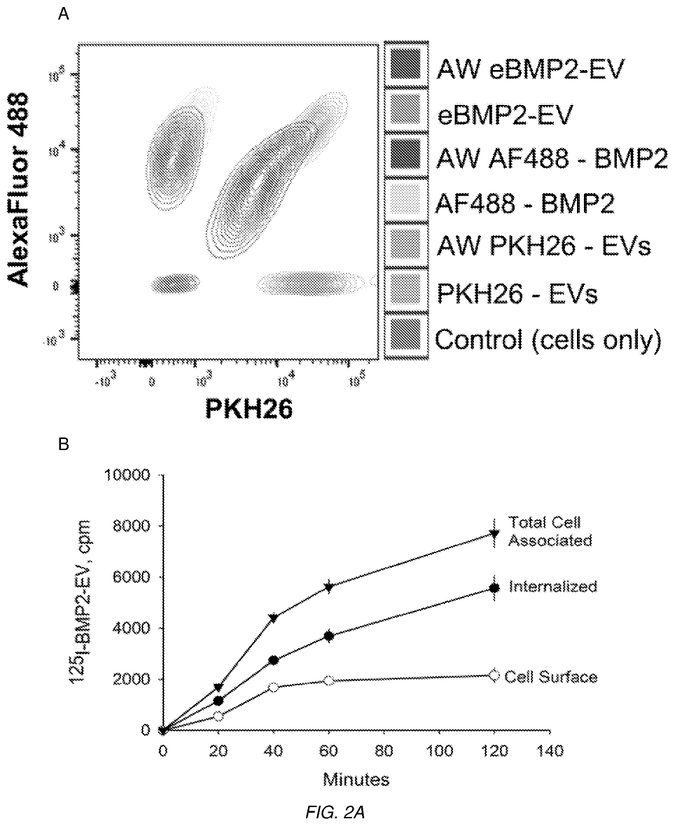

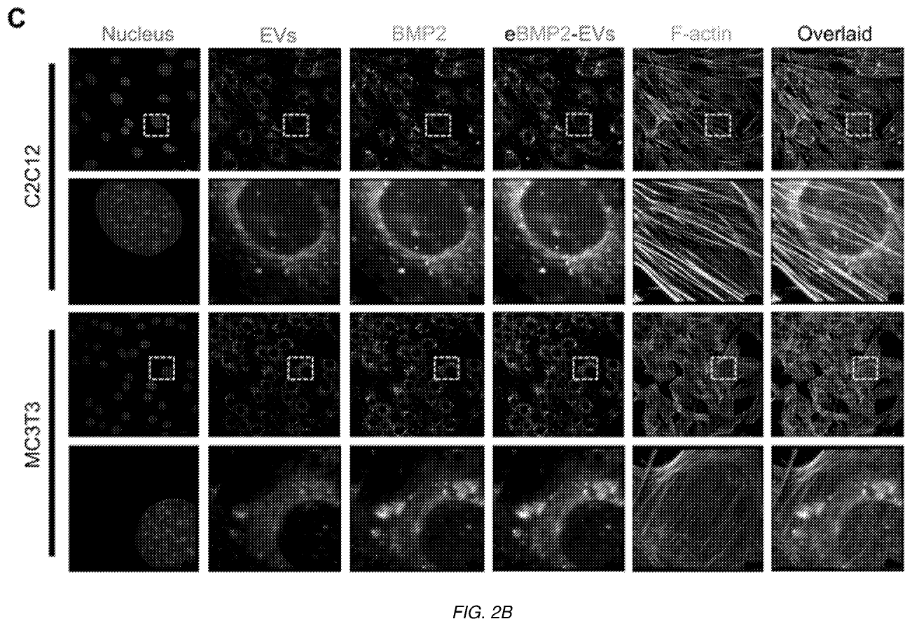

eBMP2-EVs are internalized by C2C12 and MC3T3 cells: Both fluorescence and radioactive labeling were used to determine cell internalization of eBMP2-EVs. By example, Alexa Fluor 488-BMP2 was loaded into EVs to determine cell uptake by MC3T3 cells using flow cytometry and fluorescence microscopy, and ¹²⁵I-BMP2 was loaded into EVs to evaluate eBMP2-EV uptake in C2C12 cells. Acid wash post BMP2 loading ensured that BMP2 was restricted to the EV lumen. Flow cytometry demonstrated Alexa Fluor 488-eBMP2-EVs internalization into MC3T3 cells (FIG. 2A (A)). Alexa Fluor 488-BMP2 uptake was also evaluated in C2C12 cells. At 4 hr, cells were analyzed for internalization of BMP2, EVs and eBMP2-EVs. In the contour plot shown in FIG. 2A (A), the X-axis indicates the PKH26 fluorescence (EV-label) while the Y-axis indicates the AlexaFluor 488 fluorescence (BMP2-label). For each, BMP2, EVs and eBMP2-EVs, approximately 30% was surface bound to MC3T3 cells at 4 hr, which was washed-off during acid rinsing. When ¹²⁵I-eBMP2-EVs were incubated with C2C12 cells, cell surface-bound ¹²⁵I-eBMP2-EVs became saturated within 45 min, whereas internalized ¹²⁵I-eBMP2-EVs continued to accumulate inside the cell over the 2 hr incubation period (FIG. 2A (B)). All subsequent radioactive cell binding experiments were based on a 1 hr incubation. Confocal microscopy (FIG. 2B (C)) revealed that the internalized eBMP2-EVs accumulated in the cytoplasm and were concentrated perinuclearly for both C2C12 and MC3T3 cells. Overlay images at higher magnification indicated intact eBMP2-EVs, as well as “free” BMP2 alone and EVs without BMP2, suggesting in part the release of “free” BMP2 into the cytoplasmic compartment. Additional 3D confocal Z-stack movies were produced (not shown).

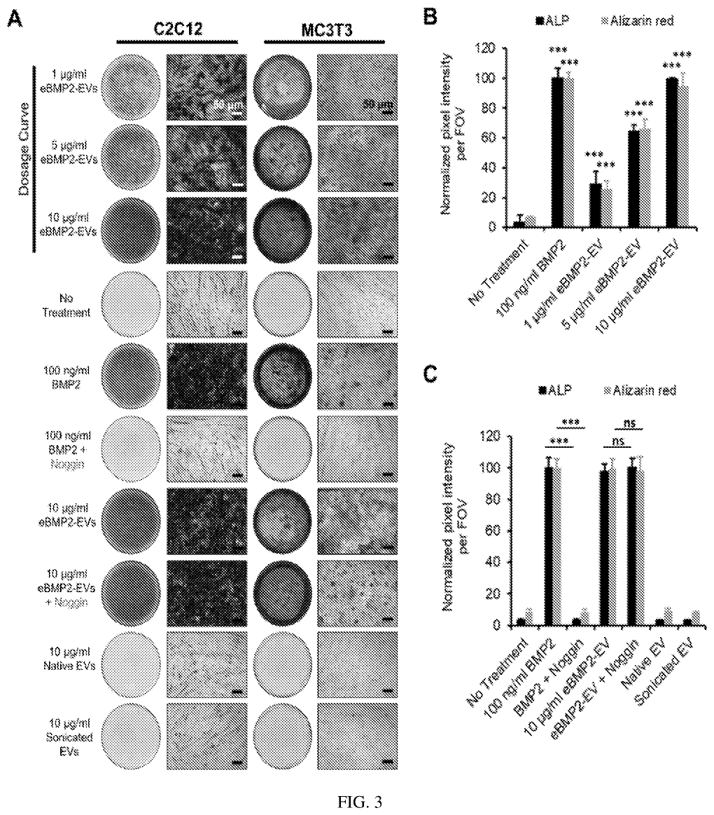

eBMP2-EVs induce osteoblastic differentiation: eBMP2-EVs applied in the liquid-phase induced cytological markers for osteoblast differentiation. An early osteogenic marker, ALP, was accessed in C2C12 cells. Mineralization, a late osteogenic marker, was accessed in MC3T3 cells using alizarin red staining for calcium. Both ALP and mineralization were induced by eBMP2-EVs (FIG. 3 (A)). Neither native non-sonicated or sonicated EVs (without BMP2) induced osteogenic differentiation. Following the quantitation of cytological staining, a dose-dependency for both ALP and mineralization was demonstrated (FIG. 3 (B,C)). The highest dose of eBMP2-EV (180 ng BMP2 in 10 pg EV protein) resulted in similar stimulation of both osteogenic markers compared to the 100 ng BMP2 positive control group.

EV intraluminal BMP2 is not subject to noggin inhibition of osteoblastic differentiation: Soluble “free” BMP2 within the extracellular microenvironment is subject to binding to various BMP2 antagonist proteins that block BMP2 interaction with its cell surface receptors, thus disrupting BMP2 signaling. Because eBMP2-EVs contain BMP2 within the EV lumen, we sought to determine if a BMP2 sequestering protein, noggin, could disrupt eBMP2-EV stimulation of osteoblast differentiation. The stimulation of both ALP and mineralization by eBMP2-EVs was found not to be inhibited by 1 μg/ml noggin, whereas “free” BMP2-mediated differentiation was inhibited (FIG. 3 (A, C)). These data demonstrate that BMP2 contained within the luminal compartment of EVs is protected from the inhibitory interaction with extracellular binding inhibitors, such as noggin.

Cell Trafficking of BMP2 in C2C12 cells: Cell trafficking experiments including recycling of cell membrane internalized growth factors has been extensively studied in regard to ¹²⁵I-epidermal growth factor (EGF), but to our knowledge EV or BMP2 cell recycling has been either minimal or not considered. We measured the C2C12 association and internalization of ¹²⁵I-BMP2 and its subsequent release into the extracellular compartment (Table 2). After 1 hr at 37° C., cell bound BMP2 was represented as ˜20% of ¹²⁵I-BMP2 associated with the cell surface while the remaining represented cell internalized ¹²⁵I-BMP2. The cell surface associated radioactivity represented BMP2 bound to both BMP2 receptors and heparin sulfate proteoglycans (HSPGs), with HSPG binding sites being the predominant type in C2C12 cells.

After acid stripping of cell surface-bound ¹²⁵I-BMP2 and following the return of stripped cells to fresh binding media at 37° C., recycling of internalized ¹²⁵I-BMP2 was determined. A total of ˜40% of internalized ¹²⁵I-BMP2 was externalized with ˜14% associated with the cell surface and the remaining released into the media. When the recycled media BMP2 was eluted using SEC, ˜82% of the ¹²⁵I-BMP2 eluted as ¹²⁵I-BMP2-EVs which we designated ¹²⁵I-nBMP2-EVs. The remaining recycled media BMP2 represented “free” BMP2.

TABLE 2
Cell Trafficking of ¹²⁵I-BMP2 in C2C12 Cells
Total Cell Associated BMP2

| | |
|---|---|
| Cell Surface % | 19.68 ± 0.72 |
| Internalized % | 80.32 ± 0.72 |

Recycled BMP2

| | |
|---|---|
| Total Recycled, % Internalized | 40.36 ± 0.50 |
| Cell Surface % | 13.78 ± 0.57 |
| Released to Media % | 86.22 ± 0.57 |

Recycled Media BMP2

| | |
|---|---|
| EV fraction % | 82.23 ± 0.58 |
| “Free” BMP2 fraction % | 13.54 ± 0.56 |

Recycled Media BMP2 EV, cargo location

| | |
|---|---|
| EV surface % | 92.01 ± 0.77 |
| EV lumen % | 7.99 ± 0.77 |

Data represent the mean±SEM of 6 replicate cultures of a representative experiment for total cell associated BMP2 and recycled BMP2. Whereas data represents the mean±SEM of 3 experiments conducted using “pooled” recycled media BMP2.

To determine the location of BMP2 as EV cargo, ¹²⁵I-nBMP2-EVs were treated with trypsin, 37° C., 30 min, followed by SEC elution. Compared to non-trypsin control groups, trypsin treatment resulted in ˜92% loss of ¹²⁵I-BMP2 from the EV fraction, suggesting that the bulk of BMP2 associated as EV cargo in nBMP2-EVs was bound to the surface. The remaining 8% was designated as intralumenal BMP2. Additional experiments were performed to access the ability of heparin to disassociate ¹²⁵I-BMP2 from ¹²⁵I-nBMP2-EVs. These experiments indicated that heparin treatment of ¹²⁵I-nBMP2-EVs for 15 min, 23° C., resulted in a 90±0.12% (mean±SEM, 3 independent experiments) loss of ¹²⁵I-BMP2 from the EV fraction, following SEC elution. This further supports that BMP2 in nBMP2-EVs is associated with the EV surface, and likely bound to glycan groups, such as HSPG on the EV surface or other EV surface proteins via similar electrostatic association.

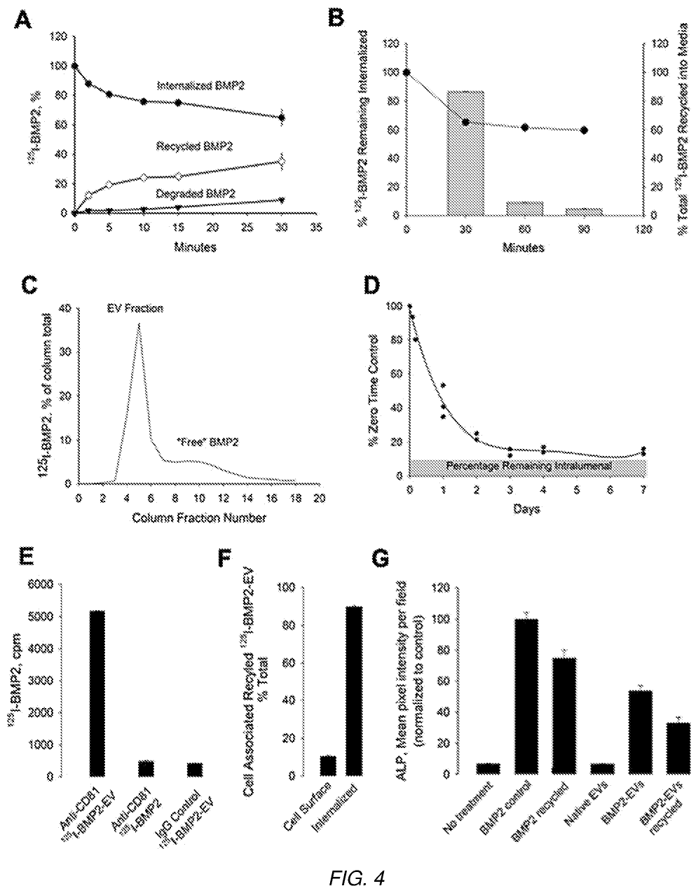

Recycling of Cell Internalized BMP2 and the generation of nBMP2-EVs: Internalized ¹²⁵I-BMP2 exhibited recycled release into media in as little as 2 min with minimal degradation throughout the sampling period (FIG. 4 (A)). Recycling occurred in essentially the first 30 min with minimal cell release occurring afterwards (FIG. 4 (B)) with ˜60% remaining internalized. The recycled media ¹²⁵I-BMP2 was pooled and samples eluted over SEC to establish distribution between EV and “free” BMP2 fractions (FIG. 4 (C)). We assessed the stability of BMP2 on the surface of ¹²⁵I-nBMP2-EVs was similar to ¹²⁵I-eBMP2-EVs in FIG. 1C (E). In direct contrast to intralumenal BMP2 in eBMP2-EVs, BMP2 associated with the surface of nBMP2-EVs became dissociated from the surface under simulated in vivo conditions (FIG. 4 (D)). Essentially 99% of the EV surface BMP2 associated with nBMP2-EVs was released after 3 days, suggesting ongoing dissociative release of “free” BMP2 from nBMP2-EVs. Such a delayed release of BMP2 from EVs could provide an extended delivery of “free” BMP2 to bind to its cell surface receptors. This is beyond the likely direct binding of BMP2, while on the surface of nBMP2-EVs, to BMP2 cell surface receptors.

Soluble ¹²⁵I-BMP2 and unlabeled BMP2 were derived from recycling experiments and evaluated as a “naturally” occurring, cell-derived form of BMP2-EV. Experiments were designed to answer the question is internalized “free” BMP2 recycled into biologically functional BMP2-EVs?Immunoprecipitation of ¹²⁵I-nBMP2-EVs using anti-CD81 antibodies confirmed that the recycled nBMP2-EV fraction likely corresponds to exosome subfraction of EVs (FIG. 4 (E)). ¹²⁵I-nBMP2-EV, representing recycled BMP2 into EVs by C2C12 cell trafficking, represents ˜66% of the soluble recycled fraction. This cell-derived form of BMP2-EV, was capable of binding to and being internalized into C2C12 cells (FIG. 4 (F)). Biological activity of both recycled nBMP2-EV and “BMP2” fractions was demonstrated using ALP assay (FIG. 4 (G)). Similar overall protein concentrations of BMP2-EVs at 25 μg/ml, both sonication loaded and recycled loaded, and “free” BMP2 at 100 ng/ml, both stock BMP2 and recycled BMP2, all resulted in significant ALP expression. The slightly lower response for both recycled nBMP2-EV and recycled “free” BMP2 compared to their respective control groups most likely reflects other protein constituents recycled along with nBMP2-EVs and “free” BMP2 contributing the total protein concentration, but also that loading differences between EV preparations do not lend themselves to direct concentration comparisons.

Cell Trafficking of eBMP2-EVs in C2C12 cells: Expanding on the cell internalization experiments presented in FIGS. 2A and 2B, ¹²⁵I-eBMP2-EVs were incubated with C2C12 cells to indicate both internalization and recycling. After 1 hr, 37° C., ˜74% of ¹²⁵I-eBMP2-EVs were internalized within cells, the remainder on the cell surface (Table 3). After the return of acid-rinsed cells to culture, after 30 min ˜24% of the internalized ¹²⁵I-eBMP2-EVs were recycled with ˜83% of the recycled ¹²⁵I-eBMP2-EVs being released into the media and the reminder associated with the cell surface. The remaining ˜76% of ¹²⁵I-BMP2 was retained intracellularly. The cell surface associated radioactivity represents EV membrane “receptors,” such as HSPGs, and likely represent receptors existing on the cell surface at the time of the initiation of the recycling incubation and/or “receptors” that have been recycled along with ¹²⁵I-eBMP2-EVs. The recycled media ¹²⁵I-eBMP2-EVs were of insufficient radioactivity, even with pooling to evaluate EV and “free” BMP2 distributions. However, using directly radiolabeled ¹²⁵I-EVs, we determined that cell trafficking of eBMP2-EVs was similar to EVs alone, with the majority of the recycled media EVs being associated the EV fraction (data not shown), suggesting recycling of intact EVs. However, whether these recycled EVs represent exocytosis of unmodified EVs or EVs modified during endosomal processing, remains unknown.

TABLE 3
Cell Trafficking of 1251-eBMP2-EVs in C2C12 Cells
Total Cell Associated eBMP2-EVs

| | |
|---|---|
| Cell Surface % | 25.09 ± 1.02 |
| Internalized % | 74.09 ± 1.02 |

Recycled BMP2

| | |
|---|---|
| Total Recycled, % Internalized | 23.81 ± 1.00 |
| Cell Surface % | 17.35 ± 0.66 |
| Released to Media % | 82.65 ± 0.66 |

Data represent the mean ± SEM of 4 experiments.

Comparing osteogenic gene and protein expression induced by “free” BMP2 and eBMP2-EVs: The data above demonstrate not only the internalization of eBMP2-EVs but also that the osteogenic potential of eBMP2-EVs is not inhibited by noggin. The absence of EV surface BMP2 and the lack of inhibition of EV intralumenal BMP2 suggest that BMP2 is internalized directly as EV intralumenal cargo, bypassing any interaction with its cell surface BMP2 receptors. This suggests that BMP2 signaling is directly initiated intracellularly. Therefore, we looked at various cytoplasmic molecular players associated with the BMP2 signaling pathway in both C2C12 and MC3T3 cells, to determine if there were differences between BMP2 signaling cascades initiated by cell surface receptors and presumably cytoplasmic receptors.

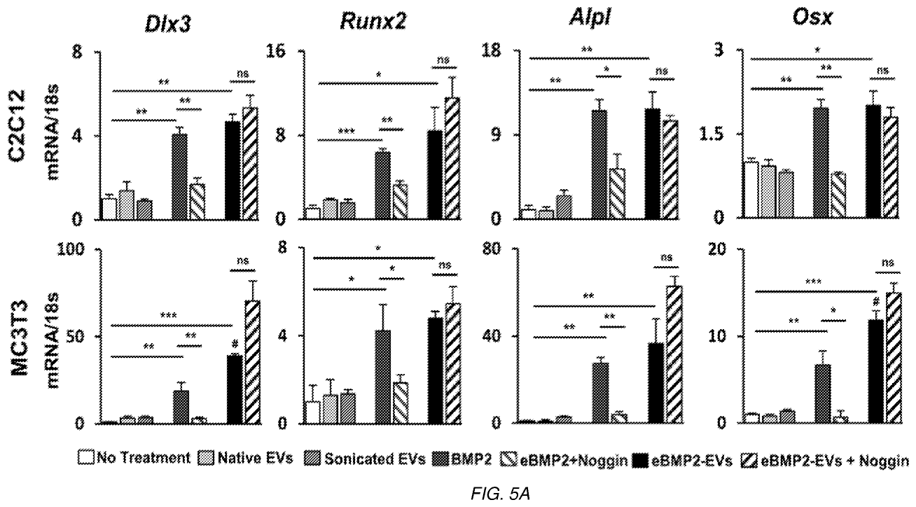

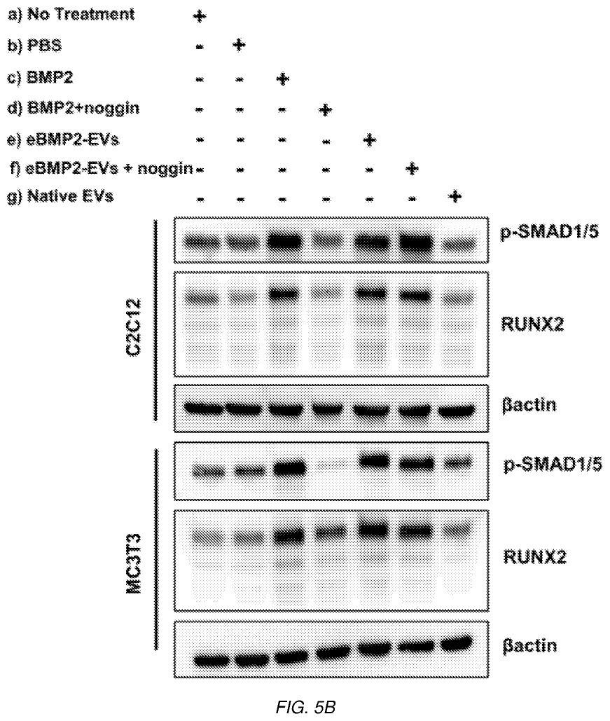

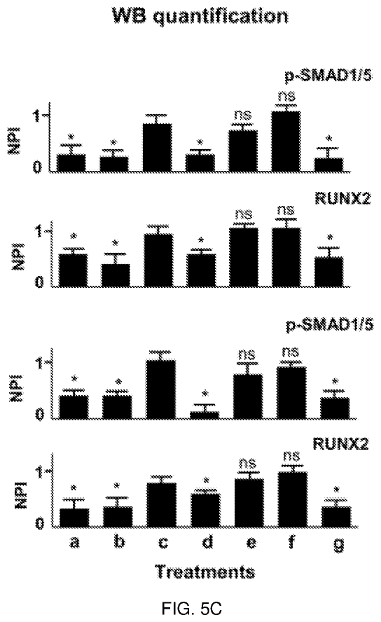

Several homeodomain (HD) proteins are critical for skeletal patterning and respond directly to BMP2 as an early step in bone formation. RUNX2, the earliest transcription factor proven essential for commitment to osteoblastogenesis, is also expressed in response to BMP2. The first set of experiments compared cell surface initiated BMP2 (100 ng/ml) and cytoplasmic initiated eBMP2-EVs (180 ng BMP/10 pg/ml EV protein), both in the absence or presence of 1 μg/ml noggin, for their ability to induce the expression level of transcripts of osteogenic genes Dlx3, Runx2, Alpl and Osx after 48 hr post-stimulation, as evaluated using qRT-PCR. These genes were selected given prior reports showing their involvement in BMP2-induced osteogenic differentiation. Both BMP2- and eBMP2-EVs-induced osteogenic gene expression for all four genes for both C2C12 and MC3T3 cells are shown (FIG. 5A). In both the cell lines, native EVs and sonicated EVs had no effect on any osteogenic genes suggesting that J774A.1 EVs do not carry native osteogenic cargo. Both BMP2 and eBMP2-EVs resulted in multi-fold increase in levels of all the osteogenic genes in both cell lines compared to no treatment control (FIG. 5A). Alpl had the most drastic (12-fold) increase in presence of BMP2 and eBMP2-EVs. In C2C12 cells there was no difference in the expression levels of all the four genes under BMP2 or eBMP2-EV stimulation. However, in MC3T3 cells, there was 20% higher expression of Dlx3 and Osx under eBMP2-EV treatment compared to BMP2 treatment. Given this is only a 0.2-fold expression difference, this may or may not hold much significance. The multi-fold increase in expression levels of all the four genes was inhibited when noggin was added along with BMP2. However, noggin did not inhibit the induction of osteogenic genes by eBMP2-EVs. These data suggest that in addition to ALP and mineralization, osteogenic gene upregulation is similarly stimulated by both BMP2 and eBMP2-EVs. Furthermore, similar to ALP and mineralization experiments, while noggin blocked gene expression for all genes stimulated by BMP2, it failed to inhibit the corresponding osteogenic gene expression induced by eBMP2-EVs.

Previous reports show that the association of RUNX2 and SMAD1/5 is essential for BMP2-induced osteogenic induction of C2C12 cells and that the physical interaction between RUNX2 and SMADs is dependent on ERK-mediated phosphorylation of RUNX2. Therefore, western blotting experiments were performed to evaluate the protein expression of pSMAD 1/5 and RUNX2 in both MC3T3 and C2C12 cells stimulated by BMP2 and eBMP2-EVs. After 72 hr exposure to BMP2 treatments, proteins were extracted for western blot analysis. Both BMP2 and eBMP2-EVs increased the protein expression of pSMAD 1/5 and RUNX2 in both cell types (FIGS. 5B and 5C). Similar to gene expression experiments, while noggin blocked the upregulation of pSMAD 1/5 and RUNX2 by BMP2, noggin did not inhibit the effect of eBMP2-EVs (FIGS. 5B and 5C).

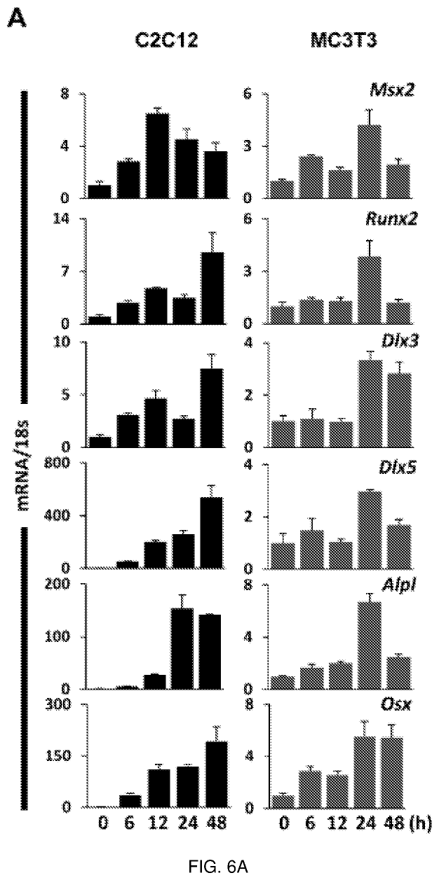

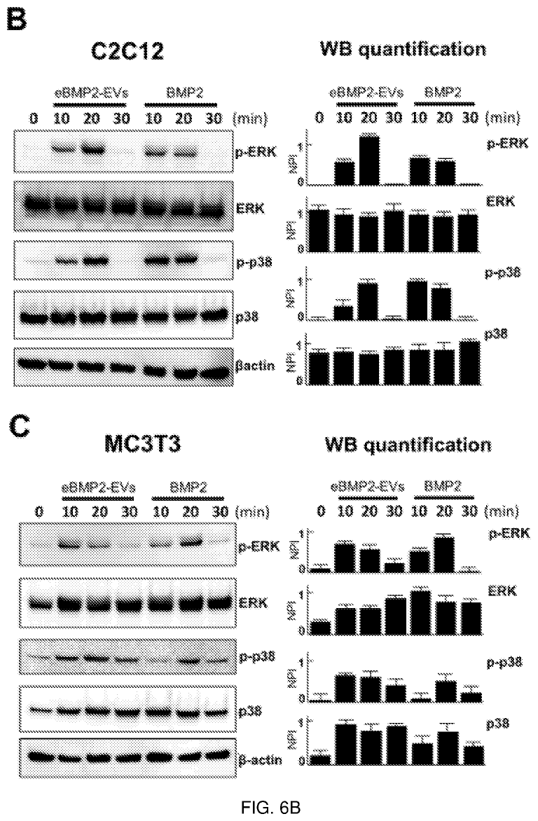

Lastly, a set of gene and western blot kinetic experiments was conducted to compare the temporal effects of BMP2 and eBMP2-EVs on the gene expression of Msx2, Runx2, Dlx3, Dlx5, Alpl and Osx. eBMP2-EVs induced time-dependent gene expression of all transcription genes in both cell types (FIG. 6A (A)). These results agree with the literature where “free” BMP2 is reported to stimulate the expression of these transcription factors in both C2C12 and MC3T3 cells. In C2C12 cells Msx2 peaked in 6 hours following which it went down, whereas in MC3T3 cells it showed a bimodal expression pattern with first peak in 6 hours followed by the second one in 24 hours. Runx2 and Dlx3 had bimodal expression patterns in C2C12s with first peak in 12 hours and a second peak appearing in 48 hours. In MC3T3 cells, Runx2 and Dlx3 were stimulated after 24 hours. Dlx5 was stimulated in 48 hours in C2C12 cells, while it showed a bimodal expression level in MC3T3 cells with a small peak in 6 hours and a second peak around 24 hours beyond which it went down. In both the cell lines, Alpl expression peaked in 24 hours after treatment but remained elevated only in C2C12s while it fell to normal levels in MC3T3 cells in 48 hours. On the other hand, Osx expression appeared to peak in 48 hours in C2C12 cells while it peaked in 24 hours in MC3T3 cells.

BMPs can signal through both canonical and non-canonical pathways. In the canonical signaling pathway, they initiate the signal transduction cascade by binding to cell surface receptors resulting in phosphorylation of SMAD. The phosphorylated SMAD proteins are then translocated to the nucleus, where they bind specific motifs in promoter regions, recruit RUNX2, and regulate the transcription of target genes. Several non-canonical, SMAD-independent signaling pathways for BMPs have been identified of which MAPK cascades are one of the most studied. In the non-canonical MAPK pathways, BMP2 activates the p38, ERK and JNK1/2 signaling pathways to promote the expression and activation of RUNX2. To compare the temporal effects of BMP2 and eBMP2-EVs on non-canonical BMP signaling pathways, phosphorylation of ERK and p38 proteins was assessed. The phosphorylation of ERK and p38 was similarly stimulated for both BMP2 and eBMP2-EVs, with phosphorylation occurring within 10 min, then reducing or ceasing by 30 min for both cell types (FIG. 6B (B, C)). Overall, the gene and protein expression levels of transcription factors involved in canonical and non-canonical BMP signaling pathways were upregulated via both BMP2 and eBMP2-EVs, which supports a broad overlap in osteogenic signaling whether initiated at the cell surface or from the cytoplasm.

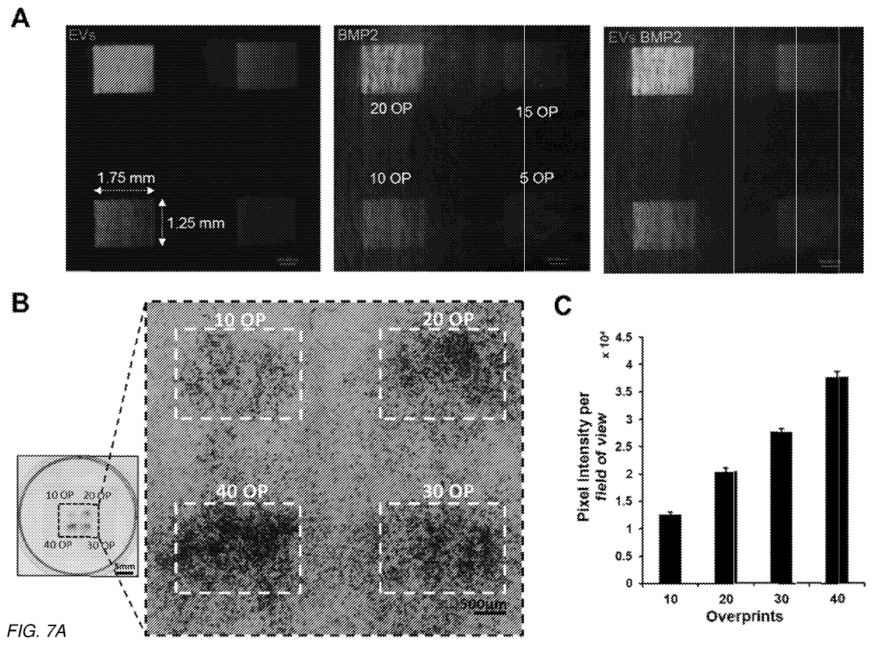

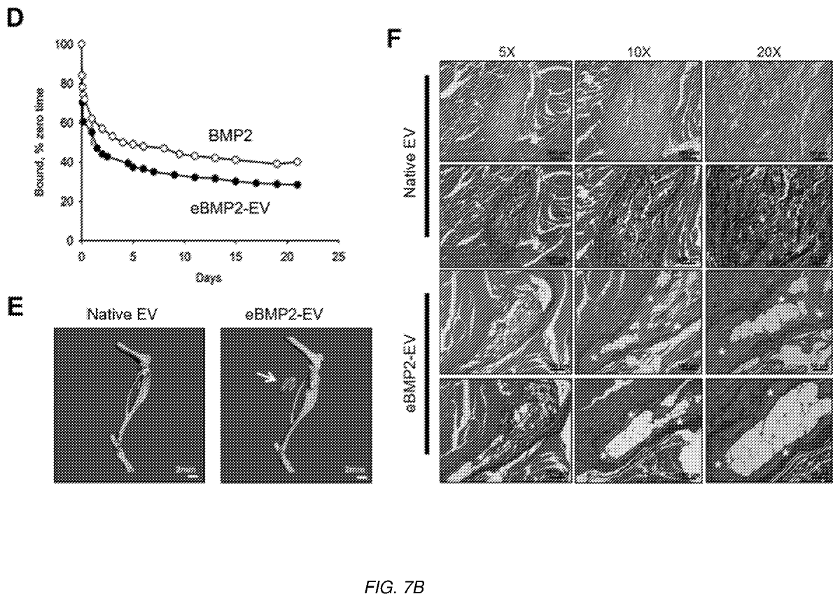

Bioprinted eBMP2-EVs microenvironments induce osteoblastogenesis in registration to printed patterns: Patterns of eBMP2-EVs (Alexa Fluor 647-labeled BMP2 and PKH26-labeled EVs) were printed onto collagen type I coated coverslips and imaged for fluorescence as shown in FIG. 7 (A). The presence of fluorescence from BMP2 and EVs after overnight rinsing in PBS demonstrated successful bioprinting and retention of eBMP2-EVs on the collagen type I coated coverslips. For in vitro biological response experiments, C2C12 cells were cultured on bioprinted eBMP2-EV microenvironments for 3 days before staining for ALP response. C2C12 cells associated with printed patterns of eBMP2-EVs differentiated toward the osteogenic lineage as evidenced by increased ALP activity (FIG. 7A (B)). ALP responsiveness was in registration to the printed eBMP2-EVs patterns and demonstrated an increase in ALP with increasing deposited concentrations of eBMP2-EVs (FIG. 7A (C)). Limited ALP response did occur off pattern immediately adjacent to pattern edges, and the intensity the off-pattern ALP response appeared directly proportional to deposited eBMP2-EV concentrations, although the vast majority of cells associated outside the spatially defined eBMP2-EVs patterns did not exhibit appreciable ALP activity and, thus, remained undifferentiated.

Bioprinted BMP2-EVs constructs locally induce heterotopic ossification (HO): 2×2 mm ADM scaffolds were bioprinted with ¹²⁵I-BMP2, ¹²⁵I-EVs or ¹²⁵I-eBMP2-EVs and their binding retention was evaluated over three weeks in SBF as shown in FIG. 7B (D). After three weeks ˜40% of ¹²⁵I-BMP2 was retained in ADM whereas ˜30% of ¹²⁵I-EV and ¹²⁵I-eBMP2-EVs were retained in ADM. Both, native EVs and eBMP2-EVs followed a similar release profile that was different from ‘free BMP2’. Printed eBMP2-EV constructs (5 ng deposited BMP2 concentration per 4.5 mm circular ADM construct) directed spatially controlled HO formation when implanted in a murine muscle pocket for 4 weeks. Radiographic μCT evaluation (FIG. 7B (E, F)) demonstrated spatially controlled HO formation at the site of eBMP2-EV construct implantation while the native EV printed ADM control construct did not. Following radiographic analysis, we performed decalcified bone histology on implant sites containing constructs and confirmed that sites of radiographic HO formation also represented histological bone (FIG. 7B (E)) as identified by H&E and Masson's trichrome staining for cells and bone morphology based on organized deposited organic matrix constituents. Decalcified histology was selected as a standard approach providing superior cell level microscopic resolution compared to un-decalcified histology and represents our standard approach for bone histological analysis.

### DISCUSSION

eBMP2-EVs were developed and characterized, and it was found that they signal cells by a novel mechanism. BMP2 cell signaling is seen to involve complex cell trafficking of BMP2, including the extracellular recycling of not only bioactive “free” BMP2, not associated with EVs, but also BMP2 bound to the surface of secreted EVs as nBMP2-EVs. nBMP2-EVs present BMP2 on the EV surface exhibiting similar cell signaling and noggin inhibition as observed for “free” BMP2. In contrast, eBMP2-EVs do not contain EV surface BMP2, therefore the intralumenal BMP2 is unable to directly interact with cell surface BMP2 receptors. However, despite its intraluminal BMP2 location, eBMP2-EVs regulated in vitro osteoblastogenesis gene/protein expression, as well as in vitro mineralization, similarly to “free” BMP2. Furthermore, eBMP2-EV regulation of osteoblastogenesis was protected from its inhibitor noggin for up to 1 month in cell culture. Lastly, eBMP2-EVs can also occur in the solid-phase (immobilized in ECM) and regulate osteoblastogenesis in register to its deposited pattern.

Macrophages play an important role in osteogenic differentiation and EVs from macrophages have been utilized to deliver exogenous biomolecules. Therefore, we decided to use murine J774A.1 cells as our source of EVs. To study osteoblastic differentiation in vitro, C2C12 and MC3T3 cells were utilized. MC3T3 represents a pre-osteoblastic origin cell line, whereas C2C12, although of myogenic origin, essentially represents a mesenchymal stem cell line. Therefore, the use of both cell lines complements each other. MC3T3 is already committed to an osteoblastic lineage, whereas C2C12 must be induced by BMP2 into an osteoblastic lineage. C2C12 cells were selected for ALP experiments because MC3T3 express a basal level of ALP in comparison to C2C12 cells, whereas MC3T3 typically give a more definitive mineralization results. In developing eBMP2-EVs, sonication of EVs in the presence of BMP2 provided the greatest loading capacity which has been prior reported as an efficient EV loading strategy for proteins (Haney M J, et al. Exosomes as drug delivery vehicles for Parkinson's disease therapy. J Control Release. 2015 Jun. 10; 207:18-30). Although the data suggested a possible ˜30% reduction in EVs post sonication (FIG. 1A (B)), the tunable resistive pulse sensing is not optimized to accurately evaluate EV concentration. More importantly, the overall size distribution profile of sonicated EVs or eBMP2-EVs (EVs sonicated in presence of BMP2) remained unchanged as compared to native EVs (non-sonicated EVs). Sonication-based loading of BMP2 into eBMP2-EVs was estimated at 18 ng BMP2/μg EV protein. This concentration of BMP EV loading far exceeds that of naturally occurring BMP2 in EVs isolated from rat growth plates at 121 pg BMP2/μg EV protein (Nahar, N. N., et al., Matrix vesicles are carriers of bone morphogenetic proteins (BMPs), vascular endothelial growth factor (VEGF), and noncollagenous matrix proteins. J Bone Miner Metab 26, 514-519 (2008) and Garimella, R., et al., Primary culture of rat growth plate chondrocytes: an in vitro model of growth plate histotype, matrix vesicle biogenesis and mineralization. Bone 34, 961-970 (2004)). It is emphasized that the BMP2 is intralumenal in eBMP2-EVs, whereas essentially all of the BMP2 is EV surface-associated in naturally occurring EVs either reported here as nBMP2-EVs or in EVs secreted during embryotic development (Draebing, T., et al., Extracellular vesicle-delivered bone morphogenetic proteins: A novel paracrine mechanism during embryonic development. bioRxiv, doi.org/10.1101/321356 (2018)). In addition, BMP2 is loaded into preformed EVs that are neutral in regard to osteogenesis (FIG. 3 (C), FIG. 4, FIGS. 7A and 7B), therefore, there is no confounding of the BMP2 osteogenic effect with other osteogenic signaling aspects such as reported in Huang et. al. Furthermore, while Huang et. al. demonstrated that EVs derived from cells engineered to overexpress BMP2 were osteogenic, they found the EVs did not contain BMP2.

Currently, BMP2 signaling is considered to be initiated upon BMP2 complexing to cell surface BMP receptors type I and II initiating intracellular signal signaling cascades via SMAD-dependent (canonical) and MAPK SMAD-independent (non-canonical) pathways that regulate osteogenic gene transcription. Internalization of the signaling BMP2-BMPR complex occurs via clathrin and caveolin endocytosis, although BMP2 signaling can persist without internalization. Upon endocytosis, the signaling BMP2-BMPR complex is incorporated into endosomes where it continues to signal intracellularly. Endosomal processing continues with either recycling of BMP2 (FIG. 4 (C)) and BMPR, or eventual degradation within the lysosome. Presumably, cell signaling of nBMP2-EVs is also initiated via direct interaction of EV surface BMP2 with cell surface BMP2 receptors and as suggested by data presented herein. Alternatively, HSPG also acts as a cell surface BMP2 binding “receptor” moiety. In C2C12 cells, HSPG BMP2 “receptors” far exceed traditional BMP2 receptor cell surface populations and compete for BMP2-BMP receptor complex formation and subsequent cell signaling, HSPG is involved in cell internalization of BMP2 and reported to act as a sink to remove extracellular “free” BMP2.

Data provided herein suggests the release of bioactive “free” BMP2 and nBMP2-EVs (FIG. 4 (C)) to the extracellular environment. Upon extracellular release, recycled “free” BMP2 is subject to immediate reassociation to cell surface BMP2 receptors or to HSPG, as well as its release into the interstitial liquid environment or immobilization within the ECM. BMP2 recycled as nBMP2-EVs can be released into the interstitial liquid environment, become immobilized within the ECM, bind to BMP2 receptors either as “free” BMP2 dissociated from nBMP2-EVs, as nBMP2-EVs, or bind to cell surface HSPG. Furthermore, recycling of the BMP2 receptors, HSPG and noggin has been proposed.

BMP2/4 are inhibited by noggin by disrupting BMP access to its cell surface BMPRs, Additionally, noggin also promotes the cellular internalization of non-signaling BMP2. This involves the formation of a binding complex of BMP-noggin-HSPG “receptors” on the cell surface, which internalize via HSPG. After internalization into perinuclear endosomes, acid-stable binding of noggin to BMP2 results in continued inhibition of BMP bioactivity. Whereas, upon internalization of BMP2 directly via HSPG, without noggin and with extracellular BMP2 removed from culture post-internalization, there is an eventual loss of internalized BMP2 which in part has been attributed to the direct transfer to immediate neighboring cells via vesicular transcytosis processes.

Considering the data provided herein, it is possible that BMP2 internalized via HSPG is recycled as both “free” BMP2 and nBMP2-EVs which may in part be responsible for paracrine signaling of neighboring cells beyond transcytosis. In the continued presence of BMP2 in the incubation media, BMP2 is internalized and remains intact. However, as these other reports did not consider BMP2 recycling it is unclear whether the internalized BMP2 represents either static loading or continuous loading, recycling and reloading. Since we only considered early loading in this study, our focus will shift to longer incubation and recycling conditions, as well as the impact of noggin, in future studies. Recycling of noggin and its involvement in intracellular BMP2 signaling also remains to be determined. Beyond BMP2-noggin-HSPG complexes with noggin serving as the binding intermediary between BMP2 and HSPG, it is possible that BMP2 also serves as the binding intermediary between noggin and HSPG. Noggin can bind and inhibit BMP2 prebound to ECM suggesting that the complex formation could potentially be initiated via BMP2 and not noggin. Lastly, noggin inhibition studies performed during this study further suggest that the release of recycled “free” BMP2 is unlikely to be involved in eBMP2-EV bioactivity, whereby the recycled “free” BMP2 binds to cell surface receptors, because the extracellular noggin remains available to sequester any non-EV encapsulated, “free” BMP2.

Based on current endocrine dogma, canonical and non-canonical cell signaling via extracellular growth factors, including BMP2, is initiated upon GF binding to responsive cell surface receptors either directly or through an intermediary molecule. However, the role of internalization of these protein-based ligands in signaling is less straightforward. Internalization of GF-receptors occurs via endocytosis, which can result in subsequent signal downregulation, signal maintenance or generation of additional signaling. Signaling via eBMP2-EV appears to bypass the cell surface BMP2 receptors. Several lines of evidence from the current study support this immediate bypassing of BMP2 surface receptors. First, EV luminal BMP2 retention confirmed not only acid pre-rinsing but also following trypsin EV surface treatment. Second, BMP2 was demonstrated to be 100% retained in eBMP2-EVs for a minimum of 24 hr. Lastly, eBMP2-EVs were not subject to noggin inhibition, even under extended culture conditions. eBMP2-EV binding and internalization are therefore likely dependent upon EV internalization mechanisms which include binding to cell surface HSPG. Post-HSPG binding, eBMP2-EVs can then be subject to internalization via CCPs and CAVs dependent endocytosis into endosomes. These internalized eBMP2-EVs in the early endosome still retain the BMP2 intralumenally, therefore even if BMP2 cell surface receptors are co-internalized, BMP2-to-BMP2 receptor interaction remains unlikely to occur.

Similarly, any endocytosed noggin remains in the lumen of the endosome, also unavailable to interact with EV luminal BMP2. However, upon acidification of the endosome, the EV membrane fuses with the endosome membrane, enabling the release of EV luminal BMP2 directly into the cytosol, while the BMP2 receptors and noggin remain in the lumen of the endosome. This is supported by our internalization experiments suggesting eBMP2-EVs appears to traffic from cell surface to the cytoplasm. Co-fluorescent staining of both EV and BMP2 suggest that internalized eBMP2-EVs remain intact but upon trafficking perinuclearly within endosomes are subject to the release of BMP2 from eBMP2-EVs. It is believed that this cytosolic BMP2 likely forms complexes with BMP Type I and II receptors within the cytosol. This results in the initiation of both canonical SMAD-dependent and independent BMP2 signaling pathways, similar to cell surface BMP2 binding to its cell surface receptors, which ultimately results in similar osteoblastic gene transcription. Data provided herein showed that eBMP2-EVs induced time-dependent transcription of Msx2, Runx2, Dlx3, Dlx5, Alpl and Osx in C2C12 and MC3T3 cells (FIGS. 6A (A) and 6B (B)). Runx2 is a downstream target of BMP signaling and is an essential transcription factor for the control of osteoblast differentiation. Previous reports have established that BMP2-induced Runx2 is mediated by Msx2, Dlx3 and Dlx5, while others have shown that BMP2 regulates Osx through Msx2 and Runx.

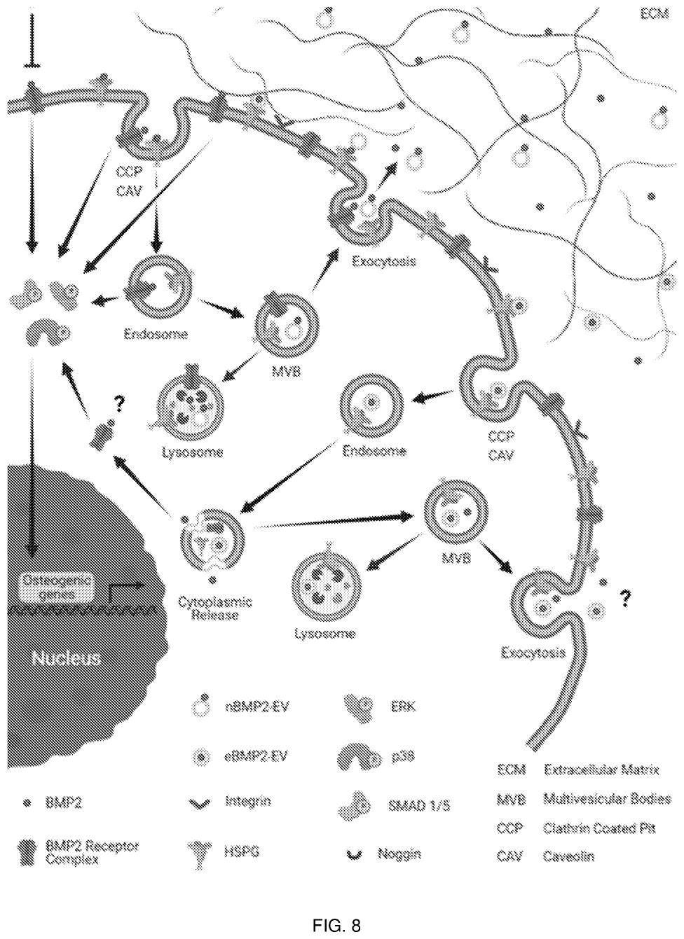

A proposed model (FIG. 8) incorporates the current literature within the context of the current study toward explaining a more complicated BMP2 signaling process involving not only BMP2 cell recycling but also EVs.

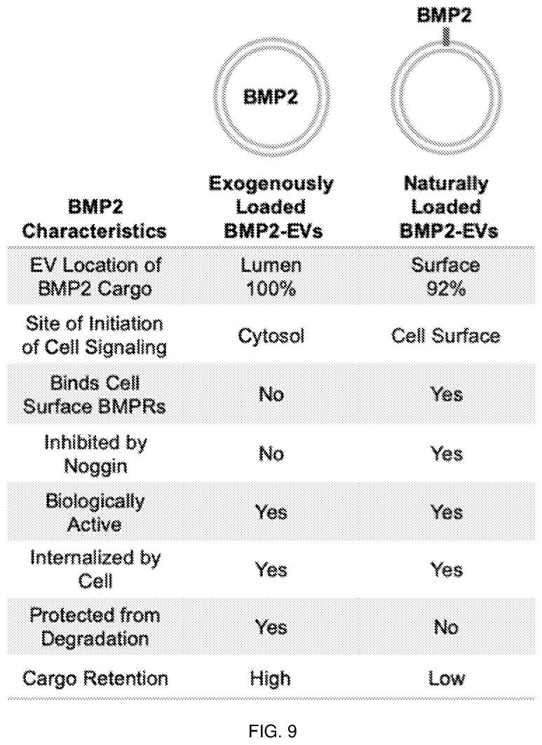

Significant differences were found between nBMP2-EVs and eBMP2-EVs, depending on where the BMP2 cargo was located. FIG. 9 directly contrasts BMP2 as EV signaling cargo, comparing eBMP2-EVs to nBMP2-EVs. The characteristics reported here for nBMP2-EVs are similar to those reported for BMP2/4-EVs in the regulation of embryonic development in zebra fish (Draebing, T., et al. (2018)). However, considering clinical therapeutic translation, the characteristics of eBMP2-EVs offer distinct advantages to nBMP2-EVs, with the potential to greatly reduce the effective dosage of delivered BMP2 without being subject to extracellular degradation or inhibition by BMP2 inhibitors.

Inkjet-based bioprinting technology was used to create eBMP2-EV solid-phase microenvironments using collagen type I-based substrates, which were tested in vitro and in vivo, demonstrating that beyond the immobilization of bioactive “free” BMP2 within the ECM, EVs can also enable immobilized reservoirs of BMP2 within the ECM. Since GFs are found in the ECM in picogram to nanogram quantities, a similar concentration range would suffice for GFs as EV cargo in the cell microenvironment. Therefore, for in vitro studies 2.4 pg to 9.6 pg (10-40 OPs) of total EV protein were printed per pattern (1.25×1.75 mm), whereas for in vivo studies approximately 280 ng EVs containing 5 ng BMP2 per construct (4.5 mm diameter disc) were printed. Although highly defined microenvironments were printed for in vitro studies, it was observed that the ALP response was not strictly confined to the printed region, where cells around the perimeter of the printed pattern also staining positive for ALP expression. One potential explanation for the ‘spillover-effect’ could be that the cells on- and off-pattern communicate in a paracrine manner affecting each other's fate, and/or it could also represent the on/off EV ECM binding kinetics that resulted in upregulation of ALP expression in vitro (FIG. 7A (B, C)), while nanogram quantities of eBMP2-EVs resulted in HO in vivo (FIG. 7B (E, F)). These results are in agreement with our previous reports on solid-phase presentation of GFs, where we demonstrated regulation of stem cell fates using picogram quantities of immobilized GFs in vitro and induction of osteogenesis in vivo using nanogram quantities of solid-phase BMP2. The purpose of the current in vivo experiment was to demonstrate that bioprinted eBMP2-EVs retained bioactivity as reflected by their ability to induce bone formation in vivo in an ectopic model. Experimental induction of ectopic bone has long been utilized in bone tissue engineering applications, including muscle pocket, subcutaneous and kidney capsule models and it remains the ‘gold standard’ in vivo bioassay to assess BMP2 bone inductive capacity. Rodent models, especially mice, are most commonly utilized for ectopic bone studies. Therefore, a murine muscle pocket model was used as a first pre-clinical demonstration of BMP2 osteoinductivity when delivered as eBMP2-EVs.

In conclusion, EVs were shown to be effective carriers for delivery of the exogenous EV intraluminal growth factor BMP2. eBMP2-EVs were internalized by cells and were biologically active, inducing osteogenic differentiation in vitro. Furthermore, cell trafficking and noggin inhibition studies collectively suggest that EV encapsulated BMP2 signaling is not initiated via binding to its cell surface receptor but results following the direct intracellular transfer of intact eBMP2-EVs. Cell trafficking experiments with “free” BMP2 suggest that nBMP2-EVs can form by innate biological processes associated with BMP2 recycling which may play a role in innate BMP2 cell signaling. However, in contrast to eBMP2-EVs, nBMP2-EVs had BMP2 bound on the EV membrane surface which was susceptible to proteolysis and heparin, as well as exhibiting cell surface receptor binding and inhibition by extracellular noggin. It is believed that this is proof of concept that other EVs with incorporated GFs, engineered similar to eBMP2-EVs, would behave similarly.

Bioprinted solid-phase eBMP2-EV microenvironments resulted in spatially controlled osteogenic differentiation in vitro using picogram-level dosages and induction of osteogenesis in vivo using nanogram-level dosages. Localized delivery of solid-phase GF-EVs via bioprinted constructs could potentially reduce the therapeutic dosages of GFs required for desired therapeutic outcomes. The ability of eEVs to facilitate potent growth factor delivery beyond that of nEV surface delivery makes them an exciting class of delivery materials with potential uses in a myriad of therapeutic applications, including tissue engineering and regenerative medicine.

Having described this invention, it will be understood to those of ordinary skill in the art that the same can be performed within a wide and equivalent range of conditions, formulations and other parameters without affecting the scope of the invention or any embodiment thereof.

1. A drug-delivery composition comprising an engineered lipid-bound vesicle comprising:
a lipid bilayer envelope defining a lumen; and
a secreted, cell surface receptor-binding signaling molecule,
wherein at least 25% of the secreted, cell surface receptor-binding signaling molecule is contained within the lumen of the lipid-bound vesicle.

2. The composition of claim 2, wherein the extracellular vesicle is an isolated exosome, nanovesicle, matrix vesicle, microparticle, or microvesicle obtained from a cell or tissue.

3. The composition of claim 2, wherein the extracellular vesicle is an isolated exosome, nanovesicle, matrix vesicle, microparticle, or microvesicle obtained from a cell or tissue.

4. The composition of claim 2, wherein the extracellular vesicle is obtained from a macrophage or a macrophage cell line.

5. The composition of claim 1, wherein the secreted, cell surface receptor-binding signaling molecule is a member of the TGF-β superfamily.

6. The composition of claim 1, wherein the secreted, cell surface receptor-binding signaling molecule is osteogenic, and optionally osteoinductive.

7. The composition of claim 6, wherein the secreted, cell surface receptor-binding signaling molecule is one or more member of a TGF-β family, optionally chosen from one of BMP1, BMP2, BMP2A, BMP3, BMP3B, BMP4, BMP5, BMP6, BMP7, BMP8A, BMP8B, BMP9, BMP10, BMP-15, and BMP heterodimers.

8. The composition of claim 6, wherein the secreted, cell surface receptor-binding signaling molecule is BMP2.

9. The composition of claim 1, wherein at least 99% of the secreted, cell surface receptor-binding signaling molecule is contained within the lumen of the lipid-bound vesicle.

10. The composition of claim 1, wherein the lumen of the lipid-bound vesicle comprises at least 1 pg, at least 10 pg, at least 100 pg, at least 500 pg, at least 1 ng, at least 2 ng, at least 3 ng, at least 4 ng, at least 5 ng, at least 6 ng, at least 7 ng, at least 8 ng, at least 9 ng, or at least 10 ng, of the secreted, cell surface receptor-binding signaling molecule per microgram (μg) of total protein of the lipid-bound vesicle.

11. The composition of claim 1, wherein the lipid-bound vesicle is stripped, for example by washing with an acid or acid buffer solution, of surface-bound secreted, cell surface receptor-binding signaling molecule.

12. The composition of claim 1, wherein the lipid-bound vesicle comprises at least two different secreted, cell surface receptor-binding signaling molecules.

13. The composition of claim 1, comprising a mixture of two different lipid-bond vesicles, each comprising a different secreted, cell surface receptor-binding signaling molecules or different amounts of the secreted, cell surface receptor-binding signaling molecules.

14. The composition of claim 1, contained within a printer cartridge or reservoir.

15. A method of preparing an engineered extracellular vesicle loaded with an isolated secreted, cell surface receptor-binding signaling molecule, comprising: sonicating a mixture of an isolated secreted, cell surface receptor-binding signaling molecule with isolated extracellular vesicles, to increase permeability of the extracellular vesicles to the secreted, cell surface receptor-binding signaling molecule, thereby loading the isolated secreted, cell surface receptor-binding signaling molecule into the lumen of the isolated extracellular vesicles, wherein the isolated extracellular vesicles or the loaded extracellular vesicles are stripped of surface-bound secreted, cell surface receptor-binding signaling molecules, for example by acid washing, wherein at least 25% of the secreted, cell surface receptor-binding signaling molecule is contained within the lumen of the loaded extracellular vesicle.

16-27. (canceled)

28. A device comprising a substrate and the composition of claim 1 coated on at least a portion of a surface of the substrate and/or incorporated into the substrate.

29. (canceled)

30. The device of claim 28, wherein the substrate comprises hydroxyapatite, and the secreted, cell surface receptor-binding signaling molecule promotes bone formation.

31. The device of claim 28, wherein the secreted, cell surface receptor-binding signaling molecule is one or more of a TGF-β superfamily ligand, a stromal cell-derived factor, an insulin-like growth factor, a vascular endothelial growth factor, a platelet-derived growth factor, and/or a member of the Interleukin family of cytokines, and is optionally chosen from one of BMP1, BMP2, BMP2A, BMP3, BMP3B, BMP4, BMP5, BMP6, BMP7, BMP8A, BMP8B, BMP9, BMP10, BMP-15, and BMP heterodimers.

32. A method of repairing or producing bone in a patient, comprising administering to the patient at a location of a bone injury or deficit an engineered lipid-bound vesicle as claimed in claim 1 in an amount effective to repair of produce bone in the patient, wherein the secreted, cell surface receptor-binding signaling molecule is osteogenic, and optionally osteoinductive.

33. The method of claim 32, wherein the secreted, cell surface receptor-binding signaling molecule comprises one or more of a TGF-β superfamily ligand, a stromal cell-derived growth factor, an insulin-like growth factor, a vascular endothelial growth factor, a platelet-derived growth factor, and/or a member of the Interleukin family of cytokines, and is optionally chosen from one of BMP1, BMP2, BMP2A, BMP3, BMP3B, BMP4, BMP5, BMP6, BMP7, BMP8A, BMP8B, BMP9, BMP10, BMP-15, and BMP heterodimers.

34-47. (canceled)


SEQUENCE LISTING (18 sequences)

SEQ ID NO: 1 - DNA, 20 bp, synthetic construct
  source 1..20: mol_type = other DNA
gagcgaccaaaggaaccata

SEQ ID NO: 2 - DNA, 20 bp, synthetic construct
  source 1..20: mol_type = other DNA
cgcttccttacctggttgat

SEQ ID NO: 3 - DNA, 20 bp, synthetic construct
  source 1..20: mol_type = other DNA
cctctgacttctgcctctgg

SEQ ID NO: 4 - DNA, 20 bp, synthetic construct
  source 1..20: mol_type = other DNA
atgaaatgcttgggaactgc

SEQ ID NO: 5 - DNA, 22 bp, synthetic construct
  source 1..22: mol_type = other DNA
tagtgaacagactccggcgcta

SEQ ID NO: 6 - DNA, 21 bp, synthetic construct
  source 1..21: mol_type = other DNA
tgtaggcggtcttcaagccat

SEQ ID NO: 7 - DNA, 25 bp, synthetic construct
  source 1..25: mol_type = other DNA
aagaagaggaagaggaagaaaatga

SEQ ID NO: 8 - DNA, 20 bp, synthetic construct
  source 1..20: mol_type = other DNA
gcttcttctccgttgtctcc

SEQ ID NO: 9 - DNA, 22 bp, synthetic construct
  source 1..22: mol_type = other DNA
agaggttcactcgctctgacga

SEQ ID NO: 10 - DNA, 21 bp, synthetic construct
  source 1..21: mol_type = other DNA
ttgctcaagtggtcgcttctg

SEQ ID NO: 11 - DNA, 21 bp, synthetic construct
  source 1..21: mol_type = other DNA
cacggccatcctatatggtaa

SEQ ID NO: 12 - DNA, 18 bp, synthetic construct
  source 1..18: mol_type = other DNA
ctgggcctggtagttgtt

SEQ ID NO: 13 - DNA, 18 bp, synthetic construct
  source 1..18: mol_type = other DNA
gtaccgggagcagccttt

SEQ ID NO: 14 - DNA, 18 bp, synthetic construct
  source 1..18: mol_type = other DNA
cttccggctcctctttca

SEQ ID NO: 15 - DNA, 18 bp, synthetic construct
  source 1..18: mol_type = other DNA
gcccctaccaccagtacg

SEQ ID NO: 16 - DNA, 18 bp, synthetic construct
  source 1..18: mol_type = other DNA
tcaccatcctcacctctg

SEQ ID NO: 17 - DNA, 20 bp, synthetic construct
  source 1..20: mol_type = other DNA
atacaggagcccggcagata

SEQ ID NO: 18 - DNA, 20 bp, synthetic construct
  source 1..20: mol_type = other DNA
cggttggtcttgtgtttcct